(19) United States
(12) Patent Application Publication
(10) Pub. No.: US 2025/0122146 A1
(43) Pub. Date: Apr. 17, 2025

(54) COMPOUNDS FOR REDUCING NEUROINFLAMMATION

(71) Applicant: THE SCRIPPS RESEARCH INSTITUTE, La Jolla, CA (US)

(72) Inventors: Stuart Lipton, Rancho Santa Fe, CA (US); Phil S. Baran, San Diego, CA (US)

(21) Appl. No.: 18/695,960

(22) PCT Filed: Sep. 27, 2022
(86) PCT No.: PCT/US22/44893

§ 371 Date: Mar. 27, 2024

Related U.S. Application Data
(60) Provisional application No. 63/261,892, filed on Sep. 30, 2021.

(51) Int. Cl. C07C 233/58; A61K 31/165; A61K 31/192; A61K 31/216; A61K 39/395; C07C 69/757

(52) U.S. Cl. CPC C07C 233/58; A61K 31/165; A61K 31/192; A61K 31/216; A61K 39/3955; C07C 69/757; C07C 2603/26

(57) ABSTRACT

Disclosed herein are compounds, their pharmaceutical compositions, and their methods of use for treating a neurodegenerative disease, such as Alzheimer's disease. Lewy body dementia, or Parkinson' disease. In some embodiments, the compound is one that activates the nuclear factor (erythroid-derived 2)-like 2 (Nrf2) and/or heat-shock factor-1 (HSF-1) transcription-mediated signaling pathway: the compound is administered with at least one antibody that is directed against an aberrant misfolded protein. The compound, illustrated by camosic acid in one example, is unexpectedly effective in reducing the type of neuroinflammation resulting from antibody-protein complexes encountered in antibody therapies of the disease. The compounds also are useful in a method of treating neuroinflammation in a subject who suffers from a neurodegenerative disease and/or has been administered at least one antibody that is directed against an aberrant misfolded protein.

### CROSS REFERENCE TO RELATED APPLICATION

This application claims priority to U.S. provisional patent application No. 63/261,892, which was filed on Sep. 30, 2021, and which is hereby incorporated by reference in its entirety

### STATEMENT OF GOVERNMENT SUPPORT

This invention was made with government support under grant number R56AG065372 awarded by the National Institutes of Health. The government has certain rights in the invention.

### BACKGROUND

The scope of protein misfolding diseases includes a range of neurodegenerative diseases. Misfolded amyloid-β peptide (Aβ) oligomers and oligomeric/fibrillar α-synuclein (αSyn) are present in several neurodegenerative disorders Other proteins that can misfold to contribute to degenerative disorders include but are not limited to tau and hyperphosphorylated tau protein, TDP-43, cystic fibrosis transmembrane conductance regulator (CFTR) protein, alpha-antitrypsin, aquaporin, huntingtin (ataxin and other trinucleotide repeats), prion, FUS, C9ORF72, ubiquilin-2, cystatin C, Notch3, GFAP, PLP, ABri, ADan, transthyretin (TTR), serpins, immunoglobulin light and heavy chains, amylin, medin, apolipoproteins AI, II and V, gelsolin, lysozyme, fibrinogen, beta-2-microglobulin, crystallins, rhodopsin, calcitonin, atrial naturetic factor (ANF), prolactin, keratoepithelin, keratins, keratin intermediate filament proteins, lactoferrin, surfactant protein C, odontogenic ameloblast-associated protein, semenogelin, apolipoproteins C2 and C3, leukocyte chemotactic factor-2, insulin, galaectin-7, corneodesmosin, enfuvirtide, and hemoglobin. Release of oligomeric/fibrillar α-synuclein (αSyn) from damaged neurons contributes to neuronal cell death partially through microglial activation in Parkinson's disease (PD) and other neurodegenerative disorders, such as Lewy body dementia (LBD), which is a form of dementia that can accompany PD (F. Bassil et al., Neuron 105, 260-275.e6 (2020); D. J. Irwin, et al. J. Alzheimers Dis. Parkinsonism 8, 444 (2018); A. R. Winslow et al., Brain 137, 1958-1970 (2014)). Microglial cells contribute to neuroinflammation, such as αSyn-activation of the NLRP3 inflammasome. In addition, the presence of Aβ oligomers exacerbates the neuroinflammation.

NLRP3 inflammasome is activated by a wide range of mechanisms, implicated in various neurological diseases, and is consequently a well-studied therapeutic target for treatment of such diseases (J. D. Lünemann et al., Ann. Neuro. 90(2) (2021) 177-188). For example, NLRP3 inflammasome plays a role in Alzheimer's disease (AD) (Mangan M S J, Olhava E J, Roush W R, Seidel H M, Glick G D, Latz E. Targeting the NLRP3 inflammasome in inflammatory diseases. Nat Rev Drug Discov. 2018 August; 17(8): 588-606. doi: 10.1038/nrd.2018.97. Epub 2018 Jul. 20. Erratum in: Nat Rev Drug Discov. 2018 September; 17(9): 688. PMID: 30026524).

Although antibody therapies have been advanced for treating protein misfolding diseases, it has been shown that αSyn-antibody complexes, especially in the presence of Aβ-antibody complexes, paradoxically trigger profound human microglial inflammatory responses (D. Trudler et al., PNAS 118(15) (2021) e2025847118). The inflammation induced by antibody-protein complexes thus magnifies neuroinflammation already present from the protein misfolding disease, frustrating antibody therapies that are intended to deplete misfolded or aggregated proteins from the human brain (id.). Because this inflammation was not seen in mouse models of disease in which prospective antibody therapies were tested, this type of inflammation was not previously known.

### SUMMARY

The deficiencies summarized above underscore a need to decrease the type and magnitude of inflammation engendered by antibody therapies of diseases whose etiologies reside in misfolded proteins such as αSyn and Aβ. The present disclosure satisfies this need and others by providing, in various embodiments, a method for treating a neurodegenerative disease in a subject suffering therefrom. The method comprises administering to the subject at least one antibody that is directed against an aberrant misfolded protein; and at least one compound or pharmaceutically acceptable salt thereof as described herein. In some embodiments, the compound is one that activates the nuclear factor (erythroid-derived 2)-like 2 (Nrf2) and/or heat-shock factor-1 (HSF-1) transcription-mediated signaling pathway. In additional embodiments, the compound is of Formula (I) or Formula (II) as described herein.

In another embodiment, the present disclosure provides a method for treating neuroinflammation in a subject suffering therefrom. The subject is one who (a) suffers from a neurodegenerative disease, and/or (b) has been administered at least one antibody that is directed against an aberrant misfolded protein. The method comprises administering to the subject at least one compound or pharmaceutically acceptable salt thereof as described herein. In some embodiments, the compound is one that activates the nuclear factor (erythroid-derived 2)-like 2 (Nrf2) and/or heat-shock factor-1 (HSF-1) transcription-mediated signaling pathway. In additional embodiments, the compound is of Formula (I) or Formula (II) as described herein.

In additional embodiments, the present disclosure provides a compound of Formula (II) or a pharmaceutically acceptable salt thereof:

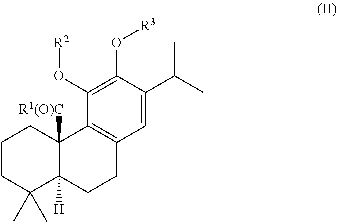

R¹ is selected from the group consisting of —NR^{A}R^{B}, —OR^{A}, —OP(O)(OR^{A})(OR^{B}).

R² and R³ are independently selected from the group consisting of H, —C(O)R^{A}, —OR^{A}, —C(O)NR^{A}R^{B}, and —OP(O)(OR^{A})(OR^{B}).

R^{A} and R^{B} are independently selected from H, C₁-C₆-alkyl, C₁-C₆-alkyl-(C₆-C₁₀-aryl);

It should be understood that, notwithstanding the definitions above, Formula (II) does not include a compound wherein R¹ is —OH and R² and R³ are simultaneously H.

Also provided in the present disclosure is a pharmaceutical composition comprising a compound or pharmaceutically acceptable salt thereof as described herein and a pharmaceutically acceptable carrier.

### BRIEF DESCRIPTION OF THE DRAWINGS

FIG. 1. Carnosic acid significantly reduced IL-1β release from human iPSC-derived microglia (hiMG) exposed to oligomeric Aβ (˜500 nM oligomers) and low concentrations of oligomeric αSyn (˜100 nM) with the respective human antibodies (anti-αSyn & anti-Aβ). Carnosic acid (0.1-10 μM) significantly attenuated IL-1β release (n=3 per group). *P<0.05, ***P<0.001, ****P<0.0001 by ANOVA with Bonferroni post hoc test.

FIG. 2. Carnosic acid significantly reduced TNF release from human iPSC-derived microglia (hiMG) exposed to oligomeric Aβ (˜750 nM oligomers) and low concentrations of oligomeric αSyn (˜100 nM) with the respective human antibodies (anti-αSyn & anti-Aβ). Carnosic acid (2 μM) significantly attenuated TNF release (n=3 per group). ****P<0.0001 by ANOVA with Bonferroni post hoc test.

### DETAILED DESCRIPTION

The present disclosure is predicated, in part, on the discovery that pro-electrophilic compounds that activate either of the Nrf2/ARE and HSF-1 transcription-mediated signaling pathways, while known to combat neuroinflammation and oxidation in the context of neurodegenerative diseases such as AD, are unexpectedly effective in reducing the type of neuroinflammation caused by antibody therapy of such diseases. The methods and compositions of the present disclosure are credited with the surprising advantage of simultaneously addressing a milieu of complex inflammatory responses operative in neurodegenerative diseases. Another advantage flowing from the present disclosure is the ability to draw from ready sources of such compounds for repurposed use in combination with new antibody therapies. In an illustrative embodiment, as demonstrated in the appended examples, the compound is carnosic acid: it is a readily available component of the herb rosemary, which is also on the U.S. FDA generally regarded as safe (GRAS) list.

### Definitions

“Alkyl” refers to straight or branched chain hydrocarbyl including from 1 to about 20 carbon atoms. For instance, an alkyl can have from 1 to 10 carbon atoms or 1 to 6 carbon atoms. Exemplary alkyl includes straight chain alkyl groups such as methyl, ethyl, propyl, butyl, pentyl, hexyl, heptyl, octyl, nonyl, decyl, undecyl, dodecyl, and the like, and also includes branched chain isomers of straight chain alkyl groups, for example without limitation, —CH(CH₃)₂, —CH(CH₃)(CH₂CH₃), —CH(CH₂CH₃)₂, —C(CH₃)₃, —C(CH₂CH₃)₃, —CH₂ CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃) CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃) CH₂CH(CH₃)₂, —CH(CH₃) CH(CH₃) CH(CH₃)₂, and the like. Thus, alkyl groups include primary alkyl groups, secondary alkyl groups, and tertiary alkyl groups. An alkyl group can be unsubstituted or optionally substituted with one or more substituents as described herein.

Each of the terms “halogen,” “halide,” and “halo” refers to —F or fluoro, —Cl or chloro, —Br or bromo, or —I or iodo.

The term “alkenyl” refers to straight or branched chain hydrocarbyl groups including from 2 to about 20 carbon atoms having 1-3, 1-2, or at least one carbon to carbon double bond. An alkenyl group can be unsubstituted or optionally substituted with one or more substituents as described herein.

“Alkyne or “alkynyl” refers to a straight or branched chain unsaturated hydrocarbon having the indicated number of carbon atoms and at least one triple bond. Examples of a (C₂-C₈)alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne and 4-octyne. An alkynyl group can be unsubstituted or optionally substituted with one or more substituents as described herein.

The term “alkoxy” or “alkoxyl” refers to an —O-alkyl group having the indicated number of carbon atoms. For example, a (C₁-C₆)-alkoxy group includes —O-methyl, —O-ethyl, —O-propyl, —O-isopropyl, —O-butyl, —O-sec-butyl, —O-tert-butyl, —O-pentyl, —O-isopentyl, —O-neopentyl, —O-hexyl, —O-isohexyl, and —O-neohexyl.

The term “cycloalkyl” refers to a saturated monocyclic, bicyclic, tricyclic, or polycyclic, 3- to 14-membered ring system, such as a C₃-C₈-cycloalkyl. The cycloalkyl may be attached via any atom. Representative examples of cycloalkyl include, but are not limited to cyclopropyl, cyclobutyl, cyclopentyl, and cyclohexyl. Polycyclic cycloalkyl includes rings that can be fused, bridged, and/or spiro-fused. A cycloalkyl group can be unsubstituted or optionally substituted with one or more substituents as described herein.

“Aryl” when used alone or as part of another term means a carbocyclic aromatic group whether or not fused having the number of carbon atoms designated or if no number is designated, up to 14 carbon atoms, such as a C₆-C₁₀-aryl or C₆-C₁₄-aryl. Examples of aryl groups include phenyl, naphthyl, biphenyl, phenanthrenyl, naphthacenyl, and the like (see, e.g., Lang's Handbook of Chemistry (Dean, J. A., ed) 13^{th} ed. Table 7-2 [1985]). “Aryl” also contemplates an aryl ring that is part of a fused polycyclic system, such as aryl fused to cycloalkyl as defined herein. An exemplary aryl is phenyl. An aryl group can be unsubstituted or optionally substituted with one or more substituents as described herein.

The term “heteroatom” refers to N, O, and S. Compounds of the present disclosure that contain N or S atoms can be optionally oxidized to the corresponding N-oxide, sulfoxide, or sulfone compounds.

“Heteroaryl,” alone or in combination with any other moiety described herein, is a monocyclic aromatic ring structure containing 5 to 10, such as 5 or 6 ring atoms, or a bicyclic aromatic group having 8 to 10 atoms, containing one or more, such as 1-4, 1-3, or 1-2, heteroatoms independently selected from the group consisting of O, S, and N. Heteroaryl is also intended to include oxidized S or N, such as sulfinyl, sulfonyl and N-oxide of a tertiary ring nitrogen. A carbon or heteroatom is the point of attachment of the heteroaryl ring structure such that a stable compound is produced. Examples of heteroaryl groups include, but are not limited to, pyridinyl, pyridazinyl, pyrazinyl, quinaoxalyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl. A heteroaryl group can be unsubstituted or optionally substituted with one or more substituents as described herein.

“Heterocycloalkyl” is a saturated or partially unsaturated non-aromatic monocyclic, bicyclic, tricyclic or polycyclic ring system that has from 3 to 14, such as 3 to 6, atoms in which 1 to 3 carbon atoms in the ring are replaced by heteroatoms of O, S or N. Polycyclic heterocycloalkyl includes rings that can be fused, bridged, and/or spiro-fused. In addition, a heterocycloalkyl is optionally fused with aryl or heteroaryl of 5-6 ring members, and includes oxidized S or N, such as sulfinyl, sulfonyl and N-oxide of a tertiary ring nitrogen. The point of attachment of the heterocycloalkyl ring is at a carbon or heteroatom such that a stable ring is retained. Examples of heterocycloalkyl groups include without limitation morpholino, tetrahydrofuranyl, dihydropyridinyl, piperidinyl, pyrrolidinyl, piperazinyl, dihydrobenzofuryl, and dihydroindolyl. A heterocycloalkyl group can be unsubstituted or optionally substituted with one or more substituents as described herein.

The term “nitrile” or “cyano” can be used interchangeably and refers to a —CN group.

The term “oxo” refers to a ═O atom bound to an atom that is part of a saturated or unsaturated moiety. Thus, the ═O atom can be bound to a carbon, sulfur, or nitrogen atom that is part of a cyclic or acyclic moiety.

A “hydroxyl” or “hydroxy” refers to an —OH group.

Compounds described herein can exist in various isomeric forms, including configurational, geometric, and conformational isomers, including, for example, cis- or trans-conformations. The compounds may also exist in one or more tautomeric forms, including both single tautomers and mixtures of tautomers. The term “isomer” is intended to encompass all isomeric forms of a compound of this disclosure, including tautomeric forms of the compound. The compounds of the present disclosure may also exist in open-chain or cyclized forms. In some cases, one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

Some compounds described herein can have asymmetric centers and therefore exist in different enantiomeric and diastereomeric forms. A compound as described herein can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

As used herein, and unless otherwise specified to the contrary, the term “compound” is inclusive in that it encompasses a compound or a pharmaceutically acceptable salt, stereoisomer, and/or tautomer thereof. Thus, for instance, a compound of Formula I includes a pharmaceutically acceptable salt of a tautomer of the compound.

In this disclosure, a “pharmaceutically acceptable salt” is a pharmaceutically acceptable, organic or inorganic acid or base salt of a compound described herein. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The terms “treat”, “treating” and “treatment” refer to the amelioration or eradication of a disease or symptoms associated with a disease. In various embodiments, the terms refer to minimizing or slowing the spread, progression, or worsening of the disease resulting from the administration of one or more prophylactic or therapeutic compounds described herein to a patient with such a disease.

The terms “prevent,” “preventing,” and “prevention” refer to the prevention of the onset, recurrence, or spread of the disease in a patient resulting from the administration of a compound described herein.

The term “effective amount” refers to an amount of a compound as described herein or other active ingredient sufficient to provide a therapeutic or prophylactic benefit in the treatment or prevention of a disease or to delay or minimize symptoms associated with a disease. Further, a therapeutically effective amount with respect to a compound as described herein means that amount of therapeutic agent alone, or in combination with other therapies, that provides a therapeutic benefit in the treatment or prevention of a disease. Used in connection with a compound as described herein, the term can encompass an amount that improves overall therapy, reduces or avoids symptoms or causes of disease, or enhances the therapeutic efficacy of or is synergistic with another therapeutic agent.

A “patient” or subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult. In the present disclosure, the terms “patient” and “subject” are used interchangeably.

### Compounds

In various embodiments, the present disclosure provides a compound of Formula (II) or a pharmaceutically acceptable salt thereof. Formula (II) compounds are useful, in some embodiments, as prodrugs of carnosic acid by virtue of one or more prodrug moieties R¹-R³, as described herein, that are readily cleaved upon exposure to a physiological milieu to yield carnosic acid:

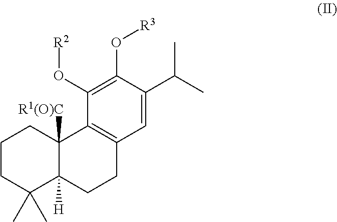

R¹ is selected from the group consisting of —NR^{A}R^{B}, —OR^{A}, —OP(O)(OR^{A})(OR^{B}).

R² and R³ are independently selected from the group consisting of H, —C(O)R^{A}, —OR^{A}, —C(O)NR^{A}R^{B}, and —OP(O)(OR^{A})(OR^{B}).

R^{A} and R^{B} are independently selected from H, C₁-C₆-alkyl, C₁-C₆-alkyl-(C₆-C₁₀-aryl). Illustrative embodiments of R^{A} and R^{B} include H, methyl, ethyl, propyl, iso-propyl, butyl, and. For example, R^{A} is H or methyl and R^{B} is H, methyl, ethyl, propyl, iso-propyl, butyl, or benzyl (Bn).

Notwithstanding the definitions of R¹, R², and R³, Formula (II) excludes a compound when R¹ is —OH and R² and R³ are simultaneously H.

In some embodiments, R² and R³ are independently —C(O)R^{A}. In other embodiments, R² and R³ are independently R^{A}. In still further embodiments, R² and R³ are independently —C(O)NR^{A}R^{B}.

In additional embodiments, optionally in combination with any other embodiment described herein, R¹ is NR^{A}R^{B}. In other embodiments, R¹ is OR^{A}.

In still additional embodiments, R¹ is —OP(O)(OR^{A})(OR^{B}). Examples of R¹ include the parent phosphate —OP(O)(OH)₂, which is further useful in the preparation of pharmaceutically acceptable salts as described herein, e.g., OP(O)(ONa)₂. Various organophosphates are contemplated, such as —OP(O)(OMe)₂.

Additional embodiments are Formula (II) compounds or their pharmaceutically acceptable salts as shown in the following table:

| | |
|---|---|
| 1 | 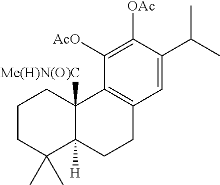 |
| 2 | 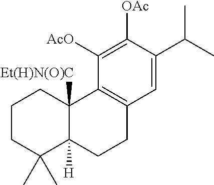 |
| 3 | 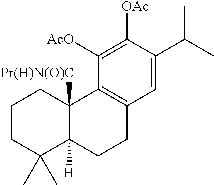 |
| 4 | 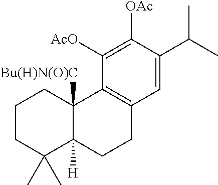 |
| 5 | 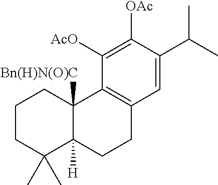 |
| 6 | 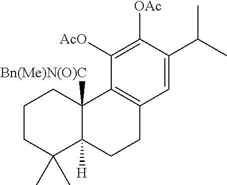 |
| 7 | 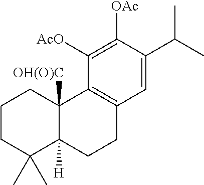 |
| 8 | 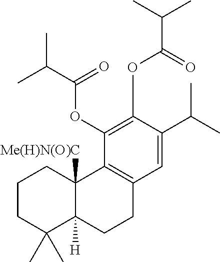 |
| 9 | 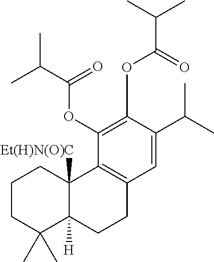 |
| 10 | 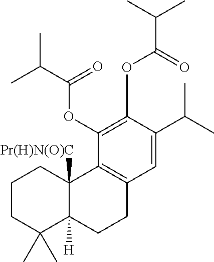 |
| 11 | 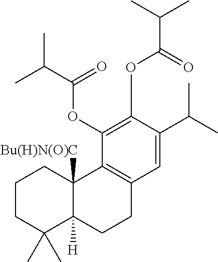 |
| 12 | 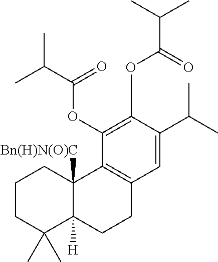 |
| 13 | 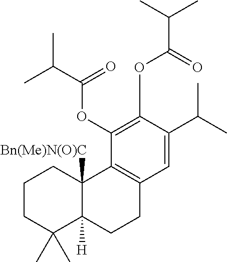 |
| 14 | 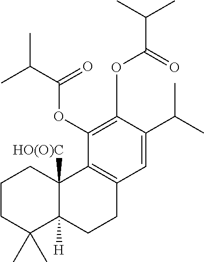 |
| 15 | 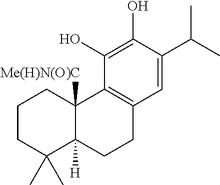 |
| 16 | 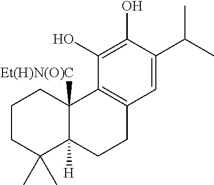 |
| 17 | 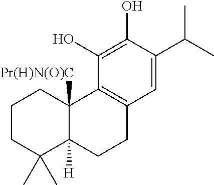 |
| 18 | 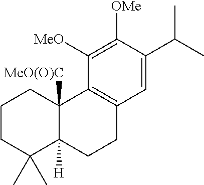 |
| 19 | 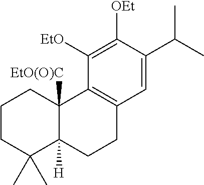 |
| 20 | 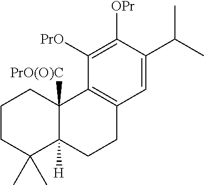 |
| 21 | 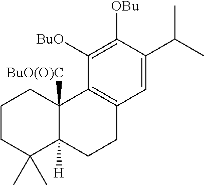 |
| 22 | 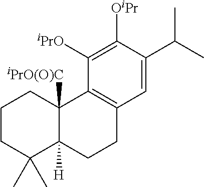 |
| 23 | 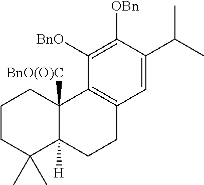 |
| 24 | 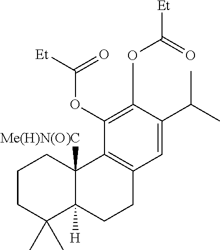 |
| 25 | 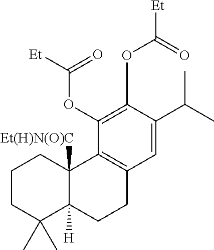 |
| 26 | 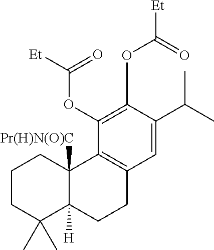 |
| 27 | 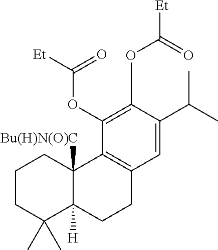 |
| 28 | 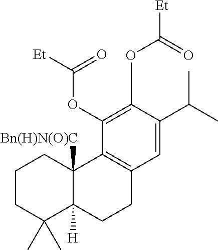 |
| 29 | 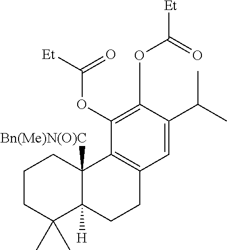 |
| 30 | 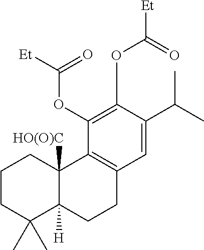 |
| 31 | 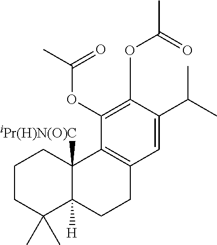 |
| 32 | 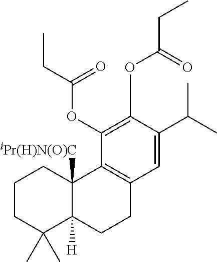 |
| 33 | 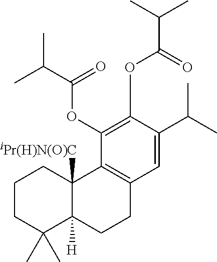 |
| 34 | 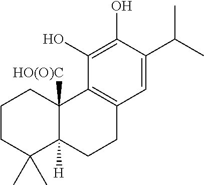 |

### Methods of Use

In various embodiments, the present disclosure provides a method for treating a neurodegenerative disease in a subject suffering therefrom. The method comprises administering to the subject:
(A) at least one antibody that is directed against an aberrant misfolded protein; and
(B) at least one compound or pharmaceutically acceptable salt thereof as described herein.

In some embodiments, the compound or pharmaceutically acceptable salt thereof is of Formula (II), as described here. In additional embodiments, the compound is one that activates the nuclear factor (erythroid-derived 2)-like 2 (Nrf2) and/or heat-shock factor-1 (HSF-1) transcription-mediated signaling pathway.

In one embodiment, the antibody (A) is administered before the compound (B). In another embodiment, the antibody (A) is administered simultaneously with the compound (B). In yet another embodiment, the antibody (A) is administered after the compound (B).

In further embodiments, the present disclosure provides a method for treating neuroinflammation in a subject suffering therefrom. In one embodiment, the subject is one who (a) suffers from a neurodegenerative disease. In another embodiment, the subject is one who (b) has been administered at least one antibody that is directed against an aberrant misfolded protein. In another embodiment, subject is characterized by both (a) and (b). In these embodiments, the method comprises administering to the subject at least one compound or pharmaceutically acceptable salt thereof, as described herein.

In various embodiments, the aberrant misfolded protein is amyloid-beta (Aβ). In other embodiments, the aberrant misfolded protein is alpha-synuclein (αSYN). In still further embodiments, the aberrant misfolded protein is a combination of Aβ and αSYN. Other embodiments include any misfolded protein against which an antibody is administered.

In various embodiments, the antibody that is administered to the subject, or that has been administered to the subject, such as in ongoing therapy, is one that is directed against an aberrant misfolded protein. In the context of neurodegenerative diseases, in exemplary embodiments, the antibody is a monoclonal antibody one directed against Aβ. Various such anti-Aβ antibodies are known in the art, including those in trials for treating Alzheimer's disease: a non-limiting list of examples includes bapineuzumab (S. Salloway, et al., N Engl J Med. 2014; 370:322-333); solanezumab (R. S. Doody, et al., N Engl J Med. 2014; 370:311-321); gantenerumab (S. Ostrowitzki S, et al., Arch Neurol. 2012; 69:198-207); crenezumab (S. Salloway, et al., Alzheimers Res Ther. 2018 Sep. 19; 10(1): 96); BAN2401 (V. Logovinsky, et al., Alzheimers Res Ther. 2016 Apr. 6; 8(1): 14); ponezumab (J. W. Landen, et al., Clin Neuropharmacol. 2013; 36:14-23); aducanumab (J. Sevigny, et al., Nature. 2016; 537:50-56); and donanemab (M. A. Mintun, et al., N Engl J Med. 2021; May 6; 384(18): 1691-1704).

In additional embodiments, the antibody is a monoclonal antibody directed against αSYN. Various anti-«SYN antibodies are known, such as in the context of treating synucleinopathies like Parkinson's disease, Lewy body dementia, and multiple system atrophy (see N. N. Vaikath et al., J. Neurochem. 150(5) (2019) 612-625; V. Gupta et al, Sci Rep 10(2020) 8137). In additional embodiments, the antibody can be against any misfolding protein contributing to disease.

### Neurodegenerative Diseases and Disorders

In the present disclosure, as contemplated in the methods disclosed herein per various embodiments, a “neurodegenerative disorder” or “neurodegenerative disease” refers to any disorder or disease of the nervous system. These include disorders and diseases that involve the central nervous system (brain, cerebrocortex, striatum, brainstem, and cerebellum), the peripheral nervous system (including cranial nerves), and the autonomic nervous system (parts of which are located in both central and peripheral nervous system). Major groups of neurodegenerative disorders and diseases include, but are not limited to, headache, stupor and coma, dementia, seizure, sleep disorders, trauma, infections, neoplasms, neuroophthalmology, movement disorders, demyelinating diseases, spinal cord disorders, and disorders of peripheral nerves, muscle, and neuromuscular junctions. Addiction and mental illness, include, but are not limited to, bipolar disorder and schizophrenia, are also included in the definition of neurodegenerative disorder.

The following is a list of several neurodegenerative disorders and diseases, symptoms, signs and syndromes that can be treated using compositions and methods according to the present disclosure: acquired epileptiform aphasia; acute disseminated encephalomyelitis; adrenoleukodystrophy; age-related macular degeneration; agenesis of the corpus callosum; agnosia; Aicardi syndrome; Alexander disease; Alpers' disease; alternating hemiplegia; Alzheimer's disease; Vascular dementia; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism and autism spectrum disorders; autonomic dysfunction; back pain; Batten disease; Behçet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome; causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy; chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease; cytomegalovirus infection; dancing eyes-dancing feet syndrome; Dandy-Walker syndrome; Dawson disease; De Morsier's syndrome; Dejerine-Klurnke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; fronto-temporal dementia; various “tauopathies”; Gaucher's disease; Gerstmann's syndrome; giant cell arteritis; giant cell inclusion disease; globoid cell leukodystrophy; Guillain-Barre syndrome; HTLV-1-associated myelopathy; Hallervorden-Spatz disease; head injury; headache; hemifacial spasm; hereditary spastic paraplegia; heredopathia atactica polyneuritiformis (Refsum's disease or phytanic acid storage disease); herpes zoster oticus; herpes zoster; Hirayama syndrome; HIV-associated dementia and neuropathy (also neurological manifestations of AIDS); holoprosencephayl; Huntington's disease and other polyglutamine repeat diseases; hydranencephaly; hydrocephalus; hypercortisolism; hypoxia; immune-mediated encephalomyelitis; inclusion body myositis; incontinentia pigrnenti; infantile phytanic acid storage disease; infantile or adult Refsum disease; infantile spasms; inflammatory myopathy; intracranial cyst; intracranial hypertension; Joubert syndrome; Kearns-Sayre syndrome; Kennedy disease Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; Lissencephaly; locked-in syndrome; Lou Gehrig's disease (i.e., motor neuron disease or amyotrophic lateral sclerosis); lumbar disc disease; Lyme disease—neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neuron disease; Moyamoya disease; mucopolysaccharidoses; milti-infarct dementia; multifocal motor neuropathy; multiple sclerosis and other demyelinating disorders; multiple system atrophy with postural hypotension; p muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenital; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; post-polio syndrome; postherpetic neuralgia; postinfectious encephalomyelitis; postural hypotension; Prader-Willi syndrome; primary lateral sclerosis; prion diseases; progressive hemifacial atrophy; progressive multifocal leukoencephalopathy; progressive sclerosing poliodystrophy; progressive supranuclear palsy; pseudotumor cerebri; Ramsay-Hunt syndrome (types I and II); Rasmussen's encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; Stiff-Person syndrome; stroke; Sturge-Weber syndrome; subacute sclerosing panencephalitis; subcortical arteriosclerotic encephalopathy; Sydenham chorea; syncope; syringomyelia; tardive dyskinesia; Tay-Sachs disease; temporal arteritis; tethered spinal cord syndrome; Thomsen disease; thoracic outlet syndrome; Tic Douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau disease; Wallenberg's syndrome; Werdnig-Hoffman disease; West syndrome; whiplash; Williams syndrome; Wilson's disease; and Zellweger syndrome.

In some embodiments, the neurodegenerative disease is chosen from Alzheimer's disease, Parkinson's disease, and Lewy body dementia. The neurodegenerative disease also contemplates various combinations of those disclosed herein, such as Lewy body dementia occurring in the context of Parkinson's disease or Alzheimer's disease.

The neurological disorder can be an affective disorder (e.g., depression or anxiety). As used herein, “affective disorder” or “mood disorder” refers to a variety of conditions characterized by a disturbance in mood as the main feature. If mild and occasional, the feelings may be normal. If more severe, they may be a sign of a major depressive disorder or dysthymic reaction or be symptomatic of bipolar disorder. Other mood disorders may be caused by a general medical condition. See, e.g., Mosby's Medical, Nursing & Allied Health Dictionary, 5th edition (1998).

As used herein, “depression” refers to an abnormal mood disturbance characterized by feelings of sadness, despair, and discouragement. Depression refers to an abnormal emotional state characterized by exaggerated feelings of sadness, melancholy, dejection, worthlessness, emptiness, and hopelessness, that are inappropriate and out of proportion to reality. See Mosby's Medical, Nursing & Allied Health Dictionary, 5th edition (1998). Depression includes, but is not limited to: a major depressive disorder (single episode, recurrent, mild, severe without psychotic features, severe with psychotic features, chronic, with catatonic features, with melancholic features, with atypical features, with postpartum onset, in partial remission, in full remission), dysthymic disorder, adjustment disorder with depressed mood, adjustment disorder with mixed anxiety and depressed mood, premenstrual dysphoric disorder, minor depressive disorder, recurrent brief depressive disorder, post-psychotic depressive disorder of schizophrenia, a major depressive disorder associated with Parkinson's disease, and a major depressive disorder associated with dementia.

The neurological disorder can be pain-associated depression (PAD). As used herein, “pain-associated depression” or “PAD” is intended to refer to a depressive disorder characterized by the co-morbidity of pain and atypical depression. Specifically, the pain can be chronic pain, neuropathic pain, or a combination thereof. Specifically, the PAD can include atypical depression and chronic pain wherein the chronic pain precedes the atypical depression, or vice versa.

“Chronic pain” refers to pain that continues or recurs over a prolonged period of time (i.e., greater than three months), caused by various diseases or abnormal conditions, such a rheumatoid arthritis, for example.

Chronic pain may be less intense than acute pain. A person with chronic pain does not usually display increased pulse and rapid perspiration because the automatic reactions to pain cannot be sustained for long periods of time. Others with chronic pain may withdraw from the environment and concentrate solely on their affliction, totally ignoring their family and friends and external stimuli. See, e.g., Mosby's Medical, Nursing & Allied Health Dictionary, 5th edition (1998).

Chronic pain includes but is not limited to: lower back pain, atypical chest pain, headache, pelvic pain, myofascial face pain, abdominal pain, and neck pain or chronic pain caused by disease or a condition such as, for example, arthritis, temporal mandibular joint dysfunction syndrome, traumatic spinal cord injury, multiple sclerosis, irritable bowel syndrome, chronic fatigue syndrome, premenstrual syndrome, multiple chemical sensitivity, closed head injury, fibromyalgia, rheumatoid arthritis, diabetes, cancer, HIV, interstitial cystitis, migraine headache, tension headache, post-herpetic neuralgia, peripheral nerve injury, causalgia, post-stroke syndrome, phantom limb syndrome e, and chronic pelvic pain.

“Atypical depression” refers to a depressed affect, with the ability to feel better temporarily in response to positive life effect (mood reactivity), plus two or more neurovegetative symptoms, including, but not limited to: hypersomnia, increased appetite or weight gain, leaden paralysis, and a long-standing pattern of extreme sensitivity to perceived interpersonal rejection; wherein the neurovegetative symptoms are present for more than about two weeks. Such neurovegetative symptoms can be reversed compared to those found in other depressive disorders (e.g., melancholic depression).

“Acute neurological disorder” refers to a neurological disorder having a rapid onset followed by a short but severe course, including, but not limited to, febrile seizures, Guillain-Barre syndrome, stroke, and intracerebral hemorrhaging.

“Chronic neurological disorder” refers to a neurological disorder lasting for a long period of time (e.g., more than about two weeks; specifically, the chronic neurological disorder can continue or recur for more than about four weeks, more than about eight weeks, or more than about twelve weeks) or is marked by frequent recurrence, including, but not limited to, narcolepsy, chronic inflammatory demyelinating polyneuropathy, cerebral palsy, epilepsy, multiple sclerosis, dyslexia, Alzheimer's disease, and Parkinson's disease.

“Trauma” refers to any injury or shock to the body, as from violence or an accident, or to any emotional wound or shock, such as a wound or shock that causes substantial, lasting damage to the psychological development of a person.

“Ischemic condition” is any condition that results in a decrease in the blood supply to a bodily organ, tissue or part caused by constriction or obstruction of the blood vessels, often resulting in a reduction of oxygen to the organ, tissue or part.

“Hypoxic conditions” are conditions in which the amount or concentration of oxygen in the air, blood or tissue is low (subnormal).

“Painful neuropathy” or “neuropathy” is chronic pain that results from damage to or pathological changes of the peripheral or central nervous system. Peripheral neuropathic pain is also referred to as painful neuropathy, nerve pain, sensory peripheral neuropathy, or peripheral neuritis. With neuropathy, the pain is not a symptom of injury but rather is itself the disease process. Neuropathy is not associated with the healing process. Rather than communicating that there is an injury somewhere, the nerves themselves malfunction and become the cause of pain.

“Neuropathic pain” refers to pain associated with inflammation or degeneration of the peripheral nerves, cranial nerves, spinal nerves, or a combination thereof. The pain is typically sharp, stinging, or stabbing. The underlying disorder can result in the destruction of peripheral nerve tissue and can be accompanied by changes in skin color, temperature, and edema. See, e.g., Mosby's Medical, Nursing & Allied Health Dictionary, 5th edition (1998); and Stedman's Medical Dictionary, 25th edition (1990).

“Diabetic neuropathy” refers to a peripheral nerve disorder/nerve damage caused by diabetes, including peripheral, autonomic, and cranial nerve disorders/damage associated with diabetes. Diabetic neuropathy is a common complication of diabetes mellitus in which nerves are damaged as a result of hyperglycemia (high blood sugar levels).

“Drug dependence” refers to habituation to, abuse of, and/or addiction to a chemical substance. Largely because of psychological craving, the life of the drug-dependent person revolves around the need for the specific effect of one or more chemical agents on mood or state of consciousness. The term thus includes not only the addiction (which emphasizes the physiological dependence) but also drug abuse (in which the pathological craving for drugs seems unrelated to physical dependence). Examples include, but are not limited to, dependence on alcohol, opiates, synthetic analgesics with morphine-like effects, barbiturates, hypnotics, sedatives, some antianxiety agents, cocaine, psychostimulants, marijuana, nicotine and psychotomimetic drugs.

“Drug withdrawal” refers to the termination of drug taking. Drug withdrawal also refers to the clinical syndrome of psychological and, sometimes, physical factors that result from the sustained use of a particular drug when the drug is abruptly withdrawn. Symptoms are variable but may include anxiety, nervousness, irritability, sweating, nausea, vomiting, rapid heart rate, rapid breathing, and seizures.

“Drug addiction” or dependence is defined as having one or more of the following signs: a tolerance for the drug (needing increased amounts to achieve the same effect), withdrawal symptoms, taking the drug in larger amounts than was intended or over a longer period of time than was intended, having a persistent desire to decrease or the inability to decrease the amount of the drug consumed, spending a great deal of time attempting to acquire the drug, or continuing to use the drug even though the person knows there are recurring physical or psychological problems caused by the drug.

“Depression” refers to a mental state of depressed mood characterized by feelings of sadness, despair, and discouragement. Depression ranges from normal feelings of the blues through dysthymia to major depression.

“Anxiety disorders” refers to an excessive or inappropriate aroused state characterized by feelings of apprehension, uncertainty, or fear. Anxiety disorders have been classified according to the severity and duration of their symptoms and specific behavioral characteristics. Categories include: generalized anxiety disorder, which is long-lasting and low-grade; panic disorder, which has more dramatic symptoms; phobias; obsessive-compulsive disorder; post-traumatic stress disorder; and separation anxiety disorder.

“Tardive dyskinesia” (e.g., Tourette's syndrome) refers to a serious, irreversible neurological disorder that can appear at any age. Tardive dyskinesia can be a side effect of long-term use of antipsychotic/neuroleptic drugs. Symptoms can be hardly noticeable or profound. Symptoms involve uncontrollable movement of various body parts, including the body, trunk, legs, arms, fingers, mouth, lips, or tongue.

“Movement disorder” refers to a group of neurological disorders that involve the motor and movement systems, including, but not limited to, ataxia, Parkinson's disease, multiple system atrophy, corticobasal degeneration, blepharospasm, Angelman syndrome, ataxia telangiectasia, dysphonia, dystonic disorders, gait disorders, torticollis, writer's cramp, progressive supranuclear palsy, Huntington's chorea, Wilson's disease, myoclonus, spasticity, tardive dyskinesia, tics, Tourette syndrome, and tremors.

“Cerebral infections that disrupt the blood-brain barrier” refers to infections of the brain or cerebrum that result in an alteration in the effectiveness of the blood-brain barrier, either increasing or decreasing its ability to prevent substances and/or organisms from passing out of the bloodstream and into the central nervous system.

“Blood-brain barrier” refers to a semi-permeable layer of endothelial cells within capillaries of the central nervous system that prevents large molecules, immune cells, many potentially damaging substances, and foreign organisms (e.g., viruses) from passing out of the bloodstream and into the central nervous system (e.g., brain and spinal cord). A dysfunction in the blood-brain barrier may underlie in part the disease process in multiple sclerosis.

“Meningitis” refers to inflammation of the meninges of the brain and spinal cord, most often caused by a bacterial or viral infection and characterized by fever, vomiting, intense headache, and stiff neck.

“Meningoencephalitis” refers to inflammation of one or both of the brain and meninges.

“Stroke,” also called cerebral accident or cerebrovascular accident, refers to a sudden loss of brain function caused by a blockage or rupture of a blood vessel to the brain (resulting in a lack of oxygen to the brain), characterized by loss of muscular control, diminution or loss of sensation or consciousness, dizziness, slurred speech, or other symptoms that vary with the extent and severity of the damage to the brain.

“Hypoglycemia” refers to an abnormally low level of glucose in the blood.

“Cerebral ischemia” (stroke) refers to a deficiency in blood supply to the brain, often resulting in a lack of oxygen to the brain.

“Cardiac arrest” refers to a sudden cessation of heartbeat and cardiac function, resulting in a temporary or permanent loss of effective circulation.

“Spinal cord trauma,” also called spinal cord injury or compression, refers to damage to the spinal cord that results from direct injury to the spinal cord itself or indirectly by damage to the bones and soft tissues and vessels surrounding the spinal cord.

“Head trauma” refers to a head injury of the scalp, skull, or brain. These injuries can range from a minor bump on the skull to a devastating brain injury. Head trauma can be classified as either closed or penetrating. In a closed head injury, the head sustains a blunt force by striking against an object. A concussion is a closed head injury that involves the brain. In a penetrating head injury, an object (usually moving at high speed, such as a windshield or other part of a motor vehicle) breaks through the skull and enters the brain.

“Perinatal hypoxia” refers to a lack of oxygen during the perinatal period (i.e., the period of time occurring shortly before and after birth, variously defined as beginning with completion of the twentieth to twenty-eighth week of gestation and ending 7 to 28 days after birth.

“Hypoglycemic neuronal damage” refers to neuronal damage, for example, nerve damage, resulting from a hypoglycemic condition (i.e., abnormally low blood glucose levels).

“Neurodegenerative disorder” refers to a type of neurological disease marked by the loss of nerve cells, including, but not limited to, Alzheimer's disease, Parkinson's disease, amyotrophic lateral sclerosis, tauopathies (including fronto-temporal dementia (FTD)), other forms of FTD due to FUS, progranulin or other mutations, and Huntington's disease and other triple repeat diseases.

“Epilepsy” refers to any of various neurological disorders characterized by sudden recurring attacks of motor, sensory, or psychic malfunction with or without loss of consciousness or convulsive seizures.

“Alzheimer's disease” refers to a disease marked by the loss of cognitive ability, generally over a period of 10 to 15 years, and associated with the development of abnormal tissues and protein deposits (Aβ plaques or tau tangles) in the cerebral cortex.

“Huntington's disease” refers to a hereditary disease that develops in adulthood and ends in dementia. It results from genetically programmed neuronal degeneration in certain areas of the brain (e.g., striatum and cortex) that causes uncontrolled movements, loss of intellectual faculties, and emotional disturbance.

“Parkinsonism” refers to a disorder similar to Parkinson's disease, but which is caused by the effects of a medication, a different neurodegenerative disorder, or another illness. The term “parkinsonism” also refers to any condition that causes any combination of the types of movement abnormalities seen in Parkinson's disease by damaging or destroying dopamine neurons in a certain area of the brain.

“Amyotrophic lateral sclerosis” (ALS), also called Lou Gehrig's disease, refers to a progressive, fatal neurological disease due to genetic mutation or sporadic etiology. ALS belongs to a class of disorders known as motor neuron disease. ALS occurs when specific nerve cells in the brain and spinal cord that control voluntary movement gradually degenerate (usually the “upper” (i.e., in the cerebrocortex) and “lower” (in the spinal cord) motor neurons. The loss of these motor neurons causes the muscles under their control to weaken and waste away, leading to paralysis. ALS manifests itself in different ways, depending on which muscles weaken first. Symptoms may include tripping and falling, loss of motor control in hands and arms, difficulty speaking, swallowing and/or breathing, persistent fatigue, and twitching and cramping, sometimes quite severely. Upper motor neuron variants (e.g., primary lateral sclerosis) are also included.

“Glaucoma” refers to any of a group of eye diseases characterized by abnormally high intraocular fluid pressure, damaged optic disk, hardening of the eyeball, and partial to complete loss of vision. The retinal ganglion cells are lost in glaucoma. Some variants of glaucoma (low tension glaucoma) have normal intraocular pressure.

“Retinal ischemia” refers to a decrease in the blood supply to the retina.

“Ischemic optic neuropathy” refers to a condition that usually presents with a sudden onset of unilaterally reduced vision. The condition is the result of decreased blood flow to the optic nerve (ischemia). There are two basic types: arteritic and non-arteritic ischemic optic neuropathy. Non-arteritic ischemic optic neuropathy is generally the result of cardiovascular disease. Patients at greatest risk have a history of high blood pressure, elevated cholesterol, smoking, diabetes, or combinations of these. Arteritic ischemic optic neuropathy is caused by the inflammation of vessels supplying blood to the optic nerves, known as temporal arteritis. This condition usually presents with sudden and severe vision loss in one eye, pain in the jaw with chewing, tenderness in the temple area, loss of appetite, and a generalized felling of fatigue or illness.

“Macular degeneration” refers to the physical disturbance of the center of the retina called the macula, leading to a loss of central vision, although color vision and peripheral vision may remain clear. Vision loss usually occurs gradually and typically affects both eyes at different rates.

A “demyelinating disorder” is a condition resulting from damage to the myelin sheath, which surrounds nerves and is responsible for efficient transmission of nerve impulses to the brain. A demyelinating disorder may result in muscle weakness, poor coordination and possible paralysis. Examples of demyelinating disorders include, but are not limited to: multiple sclerosis, optic neuritis, transverse neuritis and Guillain-Barre syndrome. When treating a demyelinating disorder, a composition according to the present invention may include an N-methyl-D-aspartate-type glutamate receptor (NMDAR) antagonist (e.g., memantine) or beta interferon isoforms, copaxone, another FDA- or EMA-approved treatment for demyelinating disease, or natalizumab or other antibody therapeutics. Since neuronal damage may occur in demyelinating conditions such as multiple sclerosis, useful drug compositions may also protect the neuron instead of or in addition to the myelin.

“Multiple sclerosis” refers to a chronic disease of the central nervous system, which predominantly affects young adults and is characterized by areas of demyelination and T-cell predominant perivascular inflammation in the white matter of the brain. Some axons may be spared from these pathological processes. The disease begins most commonly with acute or subacute onset of neurologic abnormalities. Initial and subsequent symptoms may dramatically vary in their expression and severity over the course of the disease, which usually lasts for many years. Early symptoms may include numbness and/or paresthesia, mono- or paraparesis, double vision, optic neuritis, ataxia and bladder control problems. Subsequent symptoms also include more prominent upper motor neuron signs, i.e., increased spasticity, increasing para- or quadriparesis. Vertigo, incoordination and other cerebellar problems, depression, emotional lability, abnormalities in gait, dysarthria, fatigue and pain are also commonly seen.

“Sequelae of hyperhomocystinemia” refers to a condition following as a consequence hyperhomocystinemia, i.e., elevated levels of homocysteine.

“Convulsion” refers to a violent involuntary contraction or series of contractions of the muscles.

“Pain” refers to an unpleasant sensation associated with actual or potential tissue damage that is mediated by specific nerve fibers to the brain where its conscious appreciation may be modified by various factors. See, e.g., Mosby's Medical, Nursing & Allied Health Dictionary, 5th edition (1998); and Stedman's Medical Dictionary, 25th edition (1990).

“Anxiety” refers to a state of apprehension, uncertainty, and/or fear resulting from the anticipation of a realistic or fantasized threatening event or situation, often impairing physical and psychological functioning.

“Schizophrenia” refers to any of a group of psychotic disorders usually characterized by withdrawal from reality, illogical patterns of thinking, delusions, and hallucinations, and accompanied in varying degrees by other emotional, behavioral, or intellectual disturbances. Schizophrenia is associated with dopamine imbalances in the brain and defects of the frontal lobe.

“Muscle spasm” refers to an often painful involuntary muscular contraction.

“Migraine headache” refers to a severe, debilitating headache often associated with photophobia and blurred vision.

“Urinary incontinence” refers to the inability to control the flow of urine and involuntary urination.

“Nicotine withdrawal” refers to the withdrawal from nicotine, an addictive compound found in tobacco, which is characterized by symptoms that include headache, anxiety, nausea and a craving for more tobacco. Nicotine creates a chemical dependency, so that the body develops a need for a certain level of nicotine at all times. Unless that level is maintained, the body will begin to go through withdrawal.

“Opiate tolerance” refers to a homeostatic response that reduces the sensitivity of the system to compensate for continued exposure to high levels of an opiate, e.g., heroine or morphine. When the drug is stopped, the system is no longer as sensitive to the soothing effects of the enkephalin neurons and the pain of withdrawal is produced.

“Opiate withdrawal” refers to an acute state caused by cessation or dramatic reduction of use of opiate drugs that has been heavy and prolonged (several weeks or longer). Opiates include heroin, morphine, codeine, Oxycontin, Dilaudid, methadone and others. Opiate withdrawal often includes sweating, shaking, headache, drug craving, nausea, vomiting, abdominal cramping, diarrhea, inability to sleep, confusion, agitation, depression, anxiety, and other behavioral changes.

“Emesis” refers to the act of vomiting.

“Brain edema” refers to an excessive accumulation of fluid in, on, around and/or in relation to the brain.

“AIDS- (or HIV-) induced (or associated) dementia” refers to dementia (a deterioration of intellectual faculties, such as memory, concentration, and judgment, resulting from an organic disease or disorder of the brain) induced by human immunodeficiency virus (HIV), which causes acquired immunodeficiency syndrome (AIDS), and is currently designated as neuroAIDS or HIV-associated neurocognitive disorder (HAND).

“HIV-related neuropathy” refers to a neuropathy in a mammal infected with HIV where the neuropathy is caused by HIV-1 itself or infections such as with CMV or other viruses of the herpes family. Neuropathy is the name given to a group of disorders whose symptoms may range from a tingling sensation or numbness in the toes and fingers to pain to paralysis.

“Ocular damage” refers to any damage to the eyes or in relation to the eyes. “Retinopathy” refers to any pathological disorder of the retina.

“Cognitive disorder” refers to any cognitive dysfunction, for example, disturbance of memory (e.g., amnesia) or learning.

### Neuroprotective Compounds

In some embodiments of the present disclosure, the compound that is administered pursuant to the methods described herein is a pro-electrophilic compound that, while not necessarily possessing neuroprotective properties, is converted to its active form by oxidative stress, such as is present in neurodegenerative disorders. In embodiments, the compound is an activator of the Keap1/Nrf2 (Kelch-like ECH-associated protein 1/nuclear factor erythroid 2-related factor 2) pathway, in which Nrf2 is released from the Keap1/Nrf2 complex in cytoplasm, enters the nucleus, and binds to the antioxidant response element (ARE) on promoters of phase 2 genes, induces phase 2 enzymes, and thereby provides neuroprotection against reactive oxygen species (ROS) and reactive nitrogen species (RNS) as well as inflammatory conditions. Phase 2 enzymes include hemeoxygenase-1 (HO-1), NADPH quinine oxidoreductase 1, and γ-glutamyl cysteine ligase (γ-GCL, also known as γ-glutamate cysteine ligase or γ-glutamyl cysteine ligase [γ-GCS]). These enzymes provide efficient cytoprotection, in part, by regulating the intracellular redox state (Itoh et al., Mol. Cell. Biol. 24:36-45, 2004; Gong et al., Antioxid. Redox Signal. 4:249-257, 2002). HO-1 has attracted special attention because of its therapeutic effects against neurodegenerative diseases (Maines and Panahian, in Hypoxia: From Genes to the Bedside, eds. Roach et al. (New York: Kluwer), 2001, pp. 249-272; Stocker et al., Science 235:1043-1046, 1987).

Thus, in various embodiments, the electrophilic compound is one that causes dissociation of Nrf2 from a Keap1/Nrf2 complex in a cell of a mammal, such as a neuron. In another embodiment, the electrophilic compound binds to Keap1, causing dissociation of Nrf2 from the Keap1/Nrf2 complex. According to another embodiment, the electrophilic compound increases expression of a phase 2 enzyme in the cell, including but not limited to HO-1. In another embodiment, the compound is any Nrf2 activator, activating Nrf2 either by pharmacological action or covalent reaction.

In additional embodiments, the compound or pharmaceutically acceptable salt thereof is selected as one that activates the heat-shock factor-1 (HSF-1) transcription-mediated signaling pathway. In some embodiments, the compound is one that activates both the HSF-1 and Nrf2 transcription pathways, as exemplified by carnosic acid, among others (T. Satoh et al., Nrf2 and HSF-1 Pathway Activation via Hydroquinone-Based Proelectrophilic Small Molecules Is Regulated by Electrochemical Oxidation Potential, ASN Neuro., July 2015, doi: 10.1177/1759091415593294).

In various embodiments, the compound is of Formula (1), or a pharmaceutically acceptable salt thereof:

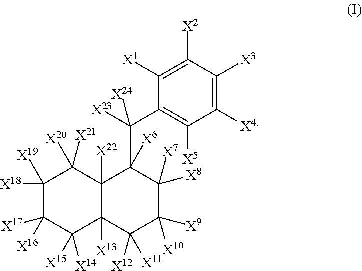

Each of X¹ to X²⁴ is independently selected from the group consisting of H, OR¹ (wherein R¹ is H or C₁-C₆-alkyl), C₁-C₆-alkyl, and W.

Each W, where present, independently is of the formula —B—C-D, —C—B-D, or —C—B—C-D.

B is selected from the group consisting of a bond, —C(O)—, —C(O)O—, —O—, —SO₂—, —NH—, —NHC(O)—, and —C(O)—NH—.

C is selected from the group consisting of a bond, and mono- or divalent C₁-C₆-alkyl, C₃-C₁₀-cycloalkyl, C₂-C₆-alkenyl, C₃-C₁₀-cycloalkenyl, C₆-C₁₀-aryl, —C₁-C₆-alkyl-(C₆-C₁₀-aryl), and —C₂-C₆-alkenyl-(C₆-C₁₀-aryl).

C is optionally substituted with one to three substituents selected from the group consisting of C₁-C₆-alkyl, C₁-C₆-haloalkyl, C₁-C₆-alkoxy, C₁-C₆-haloalkoxy, CN, OH, NO₂, —NRR′ (wherein R and R′ are independently selected from H and C₁-C₆-alkyl), —(C₁-C₆-alkyl)NRR′, —C(O)NRR′, —SO₂R, C₆-C₁₀-aryl, 3- to 6-membered heterocycloalkyl (wherein 1-4 ring members are independently selected from N, O, and S), 5- to 10-membered heteroaryl (wherein 1-4 heteroaryl members are independently selected from N, O, and S), and C₃-C₁₀-cycloalkyl.

D is selected from the group consisting of H, —C(O)OR¹, —SO₃H, —OP(O)(OR)(OR′), —NO₃, —NO₂, —NO, —NH₂, and —OH.

In some embodiments, one pair of X⁷/X⁸, X⁹/X¹⁰, X¹¹/X¹², X¹⁴/X¹⁵, X¹⁶/X¹⁷, X¹⁸/X¹⁹, X²⁰/X²¹, and X²³/X²⁴ represents ═C(R¹)₂.

In other embodiments, one pair of substituents X⁶/X⁷, X⁸/X⁹, X¹⁰/X¹¹, X¹²/X¹³, X¹³/X¹⁴, X¹⁵/X¹⁶, X¹⁷/X¹⁸, X¹⁹X²⁰, X²¹/X²², X⁶/X²², X¹³/X²², and X⁶/X²³, together with the bond between adjacent carbon atoms to which the substituents are bound, represents a double bond.

In various embodiments, two of X¹-X⁵ are —OR¹. Exemplary Formula (I) compounds, per some embodiments, are those wherein each of X¹ and X⁴ is —OR¹. In additional embodiments, each of X², X³, and X⁵ is H. In some embodiments, X¹ and X⁴ is —OR¹, and each of X², X³, and X⁵ is H. In various embodiments, each R¹ is H. All these combinations are contemplated.

Specific examples of pro-electrophilic Nrf2 transcriptional activators are now described. Many pro-electrophilic compounds suitable as Nrf2 transcriptional activators are known in the art, able to exert neuroprotective effects, and are useful in the methods described in the present disclosure. In an illustrative embodiment, the compound is carnosic acid:

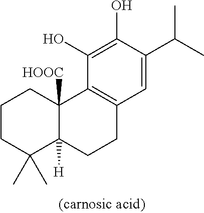

Carnosic acid is a component of the herbs rosemary and sage and is on the U.S. FDA ‘generally regarded as safe’ (GRAS) list. Carnosic acid has been shown to be a pro-electrophilic compound affording protection against neural damage, and that activates the Nrf2/ARE pathway in Alzheimer's disease (S. A. Lipton et al., Cell Death and Disease 7 (2016) e2499).

In another embodiment, and illustrative of Formula (I) compounds for use in the disclosed methods, the compound is zonarol, a terpene para-hydroquinone that is naturally occurring in brown algae:

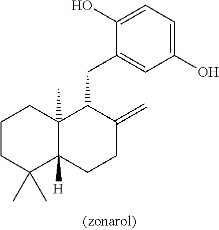

Zonarol also is shown to activate the Nrf2/ARE pathway and exert neuroprotective effects against oxidative stress (H. Shimizu et al., Biochem. Biophys. Res. Comm. 457 (2015) 718-722).

In another embodiment, the compound is yahazunol, which is also a terpene obtained from brown algae:

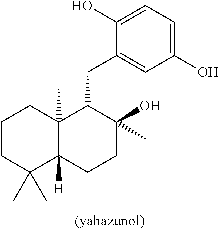

Many additional pro-electrophilic compounds, like zonarol and yahazunol, are neuroprotective terpenes readily sourced from various brown algae and sponges, per various embodiments. See, e.g., T. Laube et al., Tetrahedron 58 (2002) 4299-4309; T. Laube et al., Tetrahedron 61 (2005) 1141-1148; D. Sladic et al., Molecules 11 (2006) 1-33; M. Gordaliza, Marine Drugs 8 (2010) 2849-2870; M. Kumagai et al., Molecules 23(5) (2018) 1214. Specific examples of pro-electrophilic compounds that activate the Nrf2/ARE pathway include those described above, and in Table 1. The compounds are known in the art and are readily isolated, for example, from various marine seaweeds and algae (see G. Zatelli et al., Revista Brasileira de Farmacognosia 28(2) (2018) 243-260 and V. L. Teixeira, et al., Revista Brasileira de Farmacognosia 29(6) (2019) 735-738).

TABLE 1
Specific pro-electrophilic compounds.

Carnosic acid
Para carnosic acid
Yahazunol
(+)-yahazunol
Zonarone
Zonarol
Isozonarone
Isozonarol
Cyclozonarone
Spongiaquinone
Hyatellaquinone
20-O-Acetyl-21-hydroxy-(−)-isozonarol
Avarol
Avarone
20-O-Acetylneoavarol
Ilimaquinone
Isospongiaquinone
5-Epi-ilimaquinone
5-Epi-isospongiaquinone
Arenarol
Puupehenone
4′-Isobutylthioavarone
3′-Methoxyavarone
Metachromins A
Metachromins B
Hypochromin A diacetate
Metachromin B monoacetate
Neomarinone
Strongylophorine
Puupehedione
Puupehediol
3′,4′-Ethylenedithioavarone
Isoarenarol
Melemeleone B
2-Epi-Nakijiquinone
FrondosinA
Peyssonols A
Peyssonols B
2-Hexaprenylhydroquinone
Dimethoxypuupehenol
Isoavarone
Nakijiquinone A
Nakijiquinone B
Nakijiquinone C
Nakijiquinone D
Nakijinol
Nakijiquinone E
Nakijiquinone F
Nakijiquinone G
Nakijiquinone H
Nakijiquinone I
Nakijiquinone J
Nakijiquinone K
Nakijiquinone L
Nakijiquinone M
Nakijiquinone N
Nakijiquinone O
Nakijiquinone P
Nakijiquinone Q
Nakijiquinone R
Polyfibrospongol A
Polyfibrospongol B
19-Hydroxy-polyfibrospongol B
Smenospondiol
5-Epi-Ilimaquinone
Ilimaquinone
Glycinylilimaquinone
Smenosquinone
Smenospongidine
(+)-Epi-Smenospongidin
Smenospongiarine
(+)-Epi-Smenospongiarin
Smenospongorine
5-Epi-Smenospongorine
Smenorthoquinone
Smenospongin
Cyclosmenospongine
Avarol
Neoavarol
6′-Hydroxyavarol
6′-Acetoxyavarol
Monoacetylavarol
6′-Hydroxy-5′-acetylavarol
Diacetylavarol
2′,5′-0-(4-Bromobenzoyl)avarol
Arenarol
Avarone
Neoavarone
4′-Methoxyavarone
4′-Methoxyneoavarone
3′-Hydroxyavarone
6′-Acetoxyavarone
3′,6′-Dihydroxyavarone
6′-Hydroxy-4′-methoxyavarone
4′-Methylaminoavarone
3′,4′-(Ethylenedithio)avarone
Melemeleone A
Melemeleone B
Arenarone
Avinosol
Wiedendiol A
Wiedendiol B
Corallidyctal A
Corallidyctal B
Corallidyctal C
Corallidyctal D
Chromazonarol
(+)-Chromazonarol
8-Epi-Chromazonarol
Puupehenol
15α-methoxypuupehenol
Cyanopuupehenone
Puupehenone
Puupehenone
Puupehenione
Cyclosiphonodictyol bis-sulfate A
Mamanuthaquinone
Smenoqualone
Strongyline A
Bolinaquinone
Dehydroxybolinaquinone
Dysidine
Methoxyhalenaquinone
Fulvanin-2
Panicein A2
Panicein F1
Renierin A
Renierin B
Popolohuanone A
Popolohuanone B
Popolohuanone C
Popolohuanone D
Dipuupehenone
Bispuupehenone
Popolohuanone E
popolohuanone F

### Pharmaceutical Composition

The disclosure also provides a pharmaceutical composition comprising a therapeutically effective amount of one or more compounds as described herein or a pharmaceutically acceptable salt, stereoisomer, and/or tautomer thereof in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

In still additional embodiments, the pharmaceutical composition comprises the compound and an antibody as described herein.

The pharmaceutical composition of the present disclosure is formulated, dosed, and administered in a fashion consistent with good medical practice. Factors for consideration in this context include the particular disorder being treated, the particular subject being treated, the clinical condition of the subject, the cause of the disorder, the site of delivery of the agent, the method of administration, the scheduling of administration, and other factors known to medical practitioners.

The “therapeutically effective amount” of a compound or a pharmaceutically acceptable salt, stereoisomer, and/or tautomer thereof that is administered is governed by such considerations, and is the minimum amount necessary to cause dissociation of Nrf2 from a Keap1/Nrf2 complex in a cell of a mammal, to bind to Keap1, to increase expression of a phase 2 enzyme, to activate HSF-1, or any combination thereof. Such amount may be below the amount that is toxic to normal cells, or the subject as a whole. Generally, the initial therapeutically effective amount of a compound (or a pharmaceutically acceptable salt, stereoisomer, or tautomer thereof) of the present disclosure that is administered is in the range of about 0.01 to about 200 mg/kg or about 0.1 to about 20 mg/kg of patient body weight per day, with the typical initial range being about 0.3 to about 15 mg/kg/day. Oral unit dosage forms, such as tablets and capsules, may contain from about 0.1 mg to about 1000 mg of a compound (or a pharmaceutically acceptable salt, stereoisomer, or tautomer thereof) of the present disclosure. In another embodiment, such dosage forms contain from about 50 mg to about 500 mg of a compound (or a pharmaceutically acceptable salt, stereoisomer, or tautomer thereof) of the present disclosure. In yet another embodiment, such dosage forms contain from about 25 mg to about 200 mg of a compound (or a pharmaceutically acceptable salt, stereoisomer, or tautomer thereof) of the present disclosure. In still another embodiment, such dosage forms contain from about 10 mg to about 100 mg of a compound (or a pharmaceutically acceptable salt, stereoisomer, or tautomer thereof) of the present disclosure. In a further embodiment, such dosage forms contain from about 5 mg to about 50 mg of a compound (or a pharmaceutically acceptable salt, stereoisomer, or tautomer thereof) of the present disclosure. In any of the foregoing embodiments the dosage form can be administered once a day or twice per day.

The compositions of the present disclosure can be administered orally, topically, parenterally, by inhalation or spray or rectally in dosage unit formulations. The term parenteral as used herein includes subcutaneous injections, intravenous, intramuscular, intraperitoneal, intracisternal injection or infusion techniques. Dosing can also be transnasal (intransal administration that passes the blood-brain barrier at the cribiform plate) to enter the brain.

Suitable oral compositions as described herein include without limitation tablets, troches, lozenges, aqueous or oily suspensions, dispersible powders or granules, emulsion, hard or soft capsules, syrups or elixirs.

In another aspect, also encompassed are pharmaceutical compositions suitable for single unit dosages that comprise a compound of the disclosure or its pharmaceutically acceptable stereoisomer, salt, or tautomer and a pharmaceutically acceptable carrier.

The compositions of the present disclosure that are suitable for oral use may be prepared according to any method known to the art for the manufacture of pharmaceutical compositions. For instance, liquid formulations of the compounds of the present disclosure contain one or more agents selected from the group consisting of sweetening agents, flavoring agents, coloring agents and preserving agents in order to provide pharmaceutically palatable preparations of a compound of the present disclosure.

For tablet compositions, a compound of the present disclosure in admixture with non-toxic pharmaceutically acceptable excipients is used for the manufacture of tablets. Examples of such excipients include without limitation inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions, a compound of the present disclosure is admixed with excipients suitable for maintaining a stable suspension. Examples of such excipients include without limitation are sodium carboxymethylcellulose, methylcellulose, hydroxpropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending a compound of the present disclosure in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide a compound of the present disclosure in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the present disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation reaction products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The compounds of the present disclosure may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

The following non-limiting examples illustrate additional embodiments of the present disclosure.

### EXAMPLES

### Part I: Synthesis of Compounds

### Example 1: Synthesis of Carnosic Acid Diacetate (7)

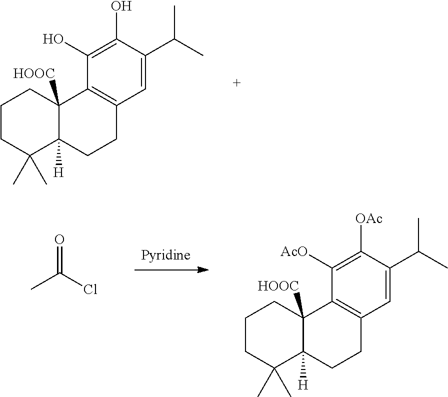

Procedure: To a stirred solution of carnosic acid (5 g, 15 mmol) in Pyridine (25 mL), was added acetyl chloride (4.72 g, 60.2 mmol) slowly. Reaction mixture was stirred for overnight. Reaction mixture was diluted with ethyl acetate, washed with 25 mL of 1N HCl solution followed by 25 mL of water and 25 mL of brine. Organic layer was dried over sodium sulfate, concentrated, and purified by silica gel column chromatography (20% to 100% ethyl acetate in petroleum ether) to get 3100 mg of pure product. 1H NMR (CDCl₃): 6.68 (s, 1H); 2.26 (s, 3H); 2.23 (s, 3H); 2.2 to 1.75 (18H); 0.96 (s, 3H); 0.82 (s, 3H). ESI-MS: M−H (negative mode)=415.2

### Example 2: Synthesis of Compound 1

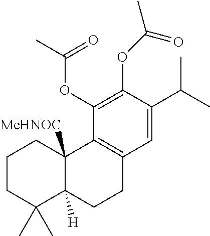

Procedure: To stirred solution of Carnosic Acid Diacetate (1) (500 mg, 1.20 mmol) in DCM (2 mL), was added oxalyl chloride and one drop of DMF. Reaction mixture was stirred at RT for 1 hr. REACTION MIXTURE was concentrated to residue which is re-dissolved in DCM (5 mL) and added drop wise to a stirred solution of Methyl amine solution (3 mL, 6.0 mmol) in DCM and TEA (0.854 mL, 6.0 mmol) at 0° C. and stirred for 1 hrs. REACTION MIXTURE was washed with water, 1N HCl solution, bicarbonate and brine solution, organic layer was dried concentrated and purified by silica gel column chromatography using a 0 to 50% ethyl acetate in petroleum ether gradient to get 130 mg of pure product. 1H NMR (CDCl₃): 6.97 (s, 1H); 5.10 (bs, 1H); 3.20 (m, 2H); 2.92 (m, 3H); 2.73 (m, 1H); 2.60 (d, 3H); 2.26 (s, 3H); 2.24 (s, 3H); 2.2-1.0 (m, 12H); 0.96 (s, 3H); 0.82 (s, 3H). ESI-MS: M+H=430.2

### Example 3: Synthesis of Compound 2

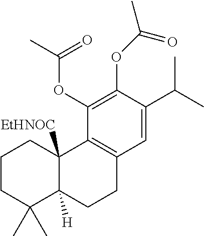

Procedure: To stirred solution of Carnosic acid diacetate (1) (500 mg, 1.20 mmol) in DCM (2 mL), was added oxalyl chloride and one drop of DMF. REACTION MIXTURE was stirred at RT for 1 hrs. REACTION MIXTURE was concentrated to residue which is dissolved in DCM again and added drop wise to a stirred solution of ethyl amine (1.85 mL, 12 mmol, 60% aqueous solution) in DCM and sodium bicarbonate (504 mg, 6.0 mmol) at 0° C. and stirred for 1 hrs. REACTION MIXTURE was washed with water, 1N HCl solution, bicarbonate and brine solution, organic layer was dried concentrated and purified by silica gel column chromatography using a 0 to 50% ethyl acetate in petroleum ether gradient to get 35 mg of pure product. 1H NMR (CDCl₃): 6.97 (s, 1H); 5.10 (bs, 1H); 3.20 (m, 2H); 2.92 (m, 3H); 2.73 (m, 1H); 2.60 (m, 2H); 2.26 (s, 3H); 2.24 (s, 3H); 2.2-1.0 (m, 15H); 0.96 (s, 3H); 0.82 (s, 3H). ESI-MS: M+H=444.2

### Example 4: Synthesis of Compound 4

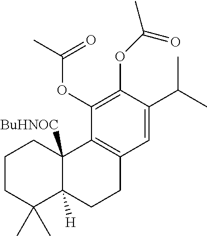

Procedure: To stirred solution of Carnosic Acid Diacetate (1) (500 mg, 1.20 mmol) in DCM (2 mL), was added oxalyl chloride and one drop of DMF. Reaction mixture was stirred at RT for 1 hr. REACTION MIXTURE was concentrated to residue which is re-dissolved in DCM (5 mL) and added drop wise to a stirred solution of Butyl amine (262 mg, 3.6 mmol) in DCM and TEA (0.854 mL, 6.0 mmol) at 0° C. and stirred for 1 hrs. REACTION MIXTURE was washed with water, 1N HCl solution, bicarbonate and brine solution, organic layer was dried concentrated and purified by silica gel column chromatography using a 0 to 50% ethyl acetate in petroleum ether gradient to get 95 mg of pure product. 1H NMR (CDCl₃): 6.97 (s, 1H); 5.10 (bs, 1H); 3.20 (m, 2H); 2.92 (m, 3H); 2.73 (m, 1H); 2.60 (m, 2H); 2.26 (s, 3H); 2.24 (s, 3H); 2.2-1.0 (m, 19H); 0.96 (s, 3H); 0.82 (s, 3H). ESI-MS: M+H=472.2

### Example 5: Synthesis of Compound 3

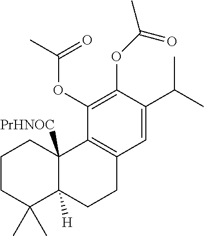

Procedure: To stirred solution of Carnosic Acid Diacetate (1) (500 mg, 1.20 mmol) in DCM (2 mL), was added oxalyl chloride and one drop of DMF. Reaction mixture was stirred at RT for 1 hr. REACTION MIXTURE was concentrated to residue which is re-dissolved in DCM (5 mL) and added drop wise to a stirred solution of propyl amine (212 mg, 3.6 mmol) in DCM and TEA (0.854 mL, 6.0 mmol) at 0° C. and stirred for 1 hrs. REACTION MIXTURE was washed with water, 1N HCl solution, bicarbonate and brine solution, organic layer was dried concentrated and purified by silica gel column chromatography using a 0 to 50% ethyl acetate in petroleum ether gradient to get 200 mg of pure product. 1H NMR (CDCl₃): 6.97 (s, 1H); 5.10 (bs, 1H); 3.20 (m, 2H); 2.92 (m, 3H); 2.73 (m, 1H); 2.60 (m, 2H); 2.26 (s, 3H); 2.24 (s, 3H); 2.2-1.0 (m, 17H); 0.96 (s, 3H); 0.82 (s, 3H). ESI-MS: M+H=459.2

### Example 6: Synthesis of Compound 5

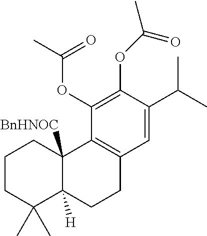

Procedure: To stirred solution of Carnosic Acid Diacetate (1) (500 mg, 1.20 mmol) in DCM (2 mL), was added oxalyl chloride and one drop of DMF. Reaction mixture (REACTION MIXTURE) was stirred at RT for 1 hr. REACTION MIXTURE was concentrated to residue which is re-dissolved in DCM (5 mL) and added drop wise to a stirred solution of Benzyl amine (385 mg, 3.6 mmol) in DCM and TEA (0.854 mL, 6.0 mmol) at 0° C. and stirred for 1 hrs. REACTION MIXTURE was washed with water, 1N HCl solution, bicarbonate and brine solution, organic layer was dried concentrated and purified by silica gel column chromatography using a 0 to 50% ethyl acetate in petroleum ether gradient to get 240 mg of pure product. 1H NMR (CDCl₃): 7.1 to 7.4 (m, 5H); 6.97 (s, 1H); 5.2 (bs, 2H); 5.10 (bs, 1H); 3.20 (m, 2H); 2.92 (m, 3H); 2.73 (m, 1H); 2.60 (m, 2H); 2.26 (s, 3H); 2.24 (s, 3H); 2.2-1.0 (m, 10H); 0.96 (s, 3H); 0.82 (s, 3H). ESI-MS: M+H=506.2.

### Example 7: Synthesis of Compound 6

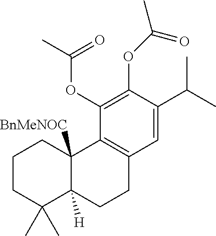

Procedure: To stirred solution of Carnosic Acid Diacetate (1) (500 mg, 1.20 mmol) in DCM (2 mL), was added oxalyl chloride and one drop of DMF. Reaction mixture (REACTION MIXTURE) was stirred at RT for 1 hr. REACTION MIXTURE was concentrated to residue which is re-dissolved in DCM (5 mL) and added drop wise to a stirred solution of N-methyl benzyl amine (436 mg, 3.6 mmol) in DCM and TEA (0.854 mL, 6.0 mmol) at 0° C. and stirred for 1 hrs. REACTION MIXTURE was washed with water, 1N HCl solution, bicarbonate and brine solution, organic layer was dried concentrated and purified by silica gel column chromatography using a 0 to 50% ethyl acetate in petroleum ether gradient to get 230 mg of pure product. 1H NMR (CDCl₃): 7.1 to 7.4 (m, 5H); 6.97 (s, 1H); 5.10 (bs, 1H); 3.20 (m, 2H); 3.15 (s, 3H); 2.92 (m, 3H); 2.73 (m, 1H); 2.60 (m, 2H); 2.26 (s, 3H); 2.24 (s, 3H); 2.2-1.0 (m, 10H); 0.96 (s, 3H); 0.82 (s, 3H). ESI-MS: M+H=520.

### Example 8: Synthesis of Compound 31

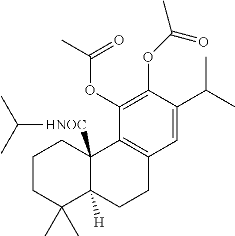

Procedure: To stirred solution of Carnosic Acid Diacetate (1)(200 mg, 0.480 mmol) in DCM (2 mL), was added oxalyl chloride (114 mg, 0.899 mmole) and one drop of DMF. Reaction mixture (REACTION MIXTURE) was stirred at RT for 1 hr. REACTION MIXTURE was concentrated to residue which is re-dissolved in DCM (5 mL) and added drop wise to a stirred solution of isopropyl amine (142 mg, 2.4 mmol) in DCM and TEA (0.344 mL, 2.40 mmol) at 0° C. and stirred for 1 hrs. REACTION MIXTURE was washed with water, 1N HCl solution, bicarbonate and brine solution, organic layer was dried concentrated and purified by silica gel column chromatography using a 0 to 50% ethyl acetate in petroleum ether gradient to get 58 mg of pure product. 1H NMR (CDCl₃): 6.97 (s, 1H); 5.10 (bs, 1H); 3.20 (m, 1H); 2.92 (m, 3H); 2.73 (m, 1H); 2.60 (m, 2H); 2.26 (s, 3H); 2.24 (s, 3H); 2.2-1.0 (m, 12H); 0.99 to 0.7 (m, 12H). ESI-MS: M+H=458.

### Example 9: Synthesis of Carnosic Acid Di-isobutyrate (14)

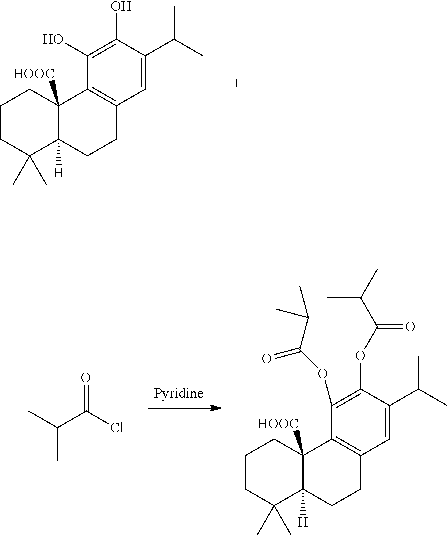

Procedure: To a stirred solution of Carnosic Acid (5000 mg, 15 mmol) in anhydrous Pyridine (25 mL), was added 4.72 g of isobutyryl chloride. The reaction mixture was stirred for 12 h at room temperature. After complete consumption of starting material, the reaction mixture was evaporated to remove 90% of pyridine, re-dissolved in ethyl acetate and washed with water (25 mL×2), saturated bicarbonate solution (25 mL×2) and brine (25 mL×1) dried over sodium sulfate, concentrated to a residue and purified by silica gel column chromatography using a 20 to 100% ethyl acetate in petroleum ether gradient to get 2800 mg of pure product. 1H NMR (CDCl₃): 6.68 (s, 1H); 2.2 to 1.5 (m, 14H); 0.9 to 0.6 (m, 24H). ESI-MS (negative mode): M−H=471

### Example 10: Synthesis of Compound 8

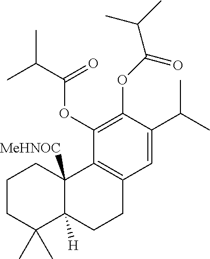

Procedure: To stirred solution of Carnosic Acid Diisobutyrate (1)(200 mg, 0.423 mmol) in DCM (2 mL), was added oxalyl chloride (0.125 g, 0.846 mmol) and one drop of DMF. Reaction mixture (REACTION MIXTURE) was stirred at RT for 1 hr. REACTION MIXTURE was concentrated to residue which is re-dissolved in DCM (5 mL) and added drop wise to a stirred solution of Methyl amine solution (1 mL, 2.0 mmol) in DCM and TEA (0.347 mL, 2.47 mmol) at 0° C. and stirred for 1 hrs. REACTION MIXTURE was washed with water, 1N HCl solution, bicarbonate and brine solution, organic layer was dried concentrated and purified by silica gel column chromatography using a 0 to 50% ethyl acetate in petroleum ether gradient to get 75 mg of pure product. 1H NMR (CDCl₃): 6.68 (s, 1H); 5.10 (bs, 1H); 2.9 (d, 3H); 2.2 to 1.5 (m, 14H); 0.9 to 0.6 (m, 24H). ESI-MS: M+H=486.

### Example 11: Synthesis of Compound 9

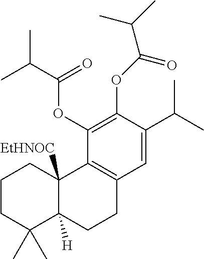

Procedure: To stirred solution of Carnosic Acid diisobutyrate (14)(500 mg, 1.20 mmol) in DCM (2 mL), was added oxalyl chloride and one drop of DMF. REACTION MIXTURE was stirred at RT for 1 hrs. REACTION MIXTURE was concentrated to residue which is dissolved in DCM again and added drop wise to a stirred solution of ethyl amine (0.7 mL, 4.23 mmol, 60% aqueous solution) in DCM and sodium bicarbonate (356 mg, 4.23 mmol) at 0° C. and stirred for 1 hr. REACTION MIXTURE was washed with water, 1N HCl solution, bicarbonate and brine solution, organic layer was dried concentrated and purified by silica gel column chromatography using a 0 to 50% ethyl acetate in petroleum ether gradient to get 75 mg of pure product. 1H NMR (CDCl₃): 6.68 (s, 1H); 5.10 (bs, 1H); 2.9 (m, 2H); 2.2 to 1.5 (m, 14H); 0.9 to 0.6 (m, 27H). ESI-MS: M+H=500.

### Example 12: Synthesis of Compound 10

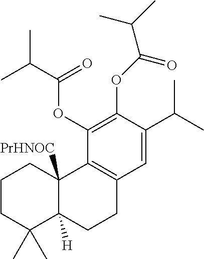

Procedure: To stirred solution of Carnosic Acid Diisobutyrate (14) (200 mg, 0.423 mmol) in DCM (2 mL), was added oxalyl chloride and one drop of DMF. Reaction mixture was stirred at RT for 1 hr. REACTION MIXTURE was concentrated to residue which is re-dissolved in DCM (5 mL) and added drop wise to a stirred solution of propyl amine (75 mg, 1.27 mmol) in DCM and TEA (0.347 mL, 2.47 mmol) at 0° C. and stirred for 1 hrs. REACTION MIXTURE was washed with water, 1N HCl solution, bicarbonate and brine solution, organic layer was dried concentrated and purified by silica gel column chromatography using a 0 to 50% ethyl acetate in petroleum ether gradient to get 80 mg of pure product. 1H NMR (CDCl): 6.68 (s, 1H); 5.10 (bs, 1H); 2.9 (m, 2H); 2.2 to 1.5 (m, 16H); 0.9 to 0.6 (m, 27H). ESI-MS: M+H=514.

### Example 13: Synthesis of Compound 33

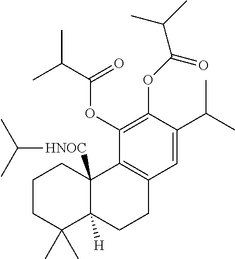

Procedure: To stirred solution of Carnosic Acid Diisobutyrate (14) (200 mg, 0.423 mmol) in DCM (2 mL), was added oxalyl chloride (114 mg, 0.846 mmol) and one drop of DMF. Reaction mixture (REACTION MIXTURE) was stirred at RT for 1 hr. REACTION MIXTURE was concentrated to residue which is re-dissolved in DCM (5 mL) and added drop wise to a stirred solution of isopropyl amine (125 mg, 2.12 mmol) in DCM and TEA (0.305 mL, 2.12 mmol) at 0° C. and stirred for 1 hrs. REACTION MIXTURE was washed with water, 1N HCl solution, bicarbonate and brine solution, organic layer was dried concentrated and purified by silica gel column chromatography using a 0 to 50% ethyl acetate in petroleum ether gradient to get 54 mg of pure product. 1H NMR (CDCl₃): 6.68 (s, 1H); 5.10 (bs, 1H); 2.9 (m, 1H); 2.2 to 1.5 (m, 14H); 0.9 to 0.6 (m, 30H). ESI-MS: M+H=514.

### Example 14: Synthesis of Compound 11

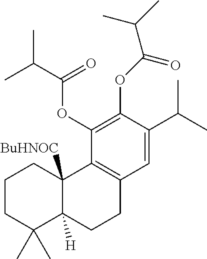

Procedure: To stirred solution of Carnosic Acid Diisobutyrate (14) (200 mg, 0.423 mmol) in DCM (2 mL), was added oxalyl chloride and one drop of DMF. Reaction mixture was stirred at RT for 1 hr. REACTION MIXTURE was concentrated to residue which is re-dissolved in DCM (5 mL) and added drop wise to a stirred solution of butyl amine (93 mg, 1.27 mmol) in DCM and TEA (0.347 mL, 2.47 mmol) at 0° C. and stirred for 1 hrs. REACTION MIXTURE was washed with water, 1N HCl solution, bicarbonate and brine solution, organic layer was dried concentrated and purified by silica gel column chromatography using a 0 to 50% ethyl acetate in petroleum ether gradient to get 90 mg of pure product. 1H NMR (CDCl₃): 6.68 (s, 1H); 5.10 (bs, 1H); 2.9 (m, 2H); 2.2 to 1.5 (m, 18H); 0.9 to 0.6 (m, 27H). ESI-MS: M+H=528.

### Example 15: Synthesis of Compound 12

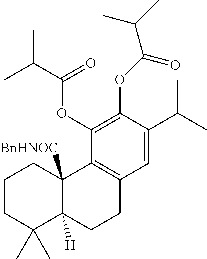

Procedure: To stirred solution of Carnosic Acid Diisobutyrate (14) (200 mg, 0.423 mmol) in DCM (2 mL), was added oxalyl chloride and one drop of DMF. Reaction mixture (REACTION MIXTURE) was stirred at RT for 1 hr. REACTION MIXTURE was concentrated to residue which is re-dissolved in DCM (5 mL) and added drop wise to a stirred solution of benzyl amine (136 mg, 1.27 mmol) in DCM and TEA (0.347 mL, 2.47 mmol) at 0° C. and stirred for 1 hrs. REACTION MIXTURE was washed with water, 1N HCl solution, bicarbonate and brine solution, organic layer was dried concentrated and purified by silica gel column chromatography using a 0 to 50% ethyl acetate in petroleum ether gradient to get 70 mg of pure product. 1H NMR (CDCl₃): 7.2 to 7.5 (m, 5H); 6.68 (s, 1H); 5.2 (bs, 2H); 5.10 (bs, 1H); 2.2 to 1.5 (m, 14H); 0.9 to 0.6 (m, 24H). ESI-MS: M+H=562.

### Example 16: Synthesis of Compound 13

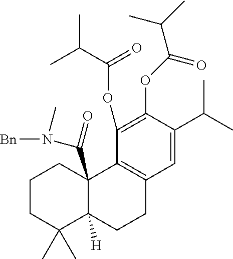 [SMILES: CC(C)C(=O)Oc1c(C(C)C)cc2c(c1OC(=O)C(C)C)[C@@]1(C(=O)N(C)Cc3ccccc3)CCCC(C)(C)[C@@H]1CC2]

Procedure: To stirred solution of Carnosic Acid Diisobutyrate (14) (200 mg, 0.423 mmol) in DCM (2 mL), was added oxalyl chloride and one drop of DMF. Reaction mixture (REACTION MIXTURE) was stirred at RT for 1 hr. REACTION MIXTURE was concentrated to residue which is re-dissolved in DCM (5 mL) and added drop wise to a stirred solution of N-methyl benzyl amine (154 mg, 1.27 mmol) in DCM and TEA (0.347 mL, 2.47 mmol) at 0° C. and stirred for 1 hrs. REACTION MIXTURE was washed with water, 1N HCl solution, bicarbonate and brine solution, organic layer was dried concentrated and purified by silica gel column chromatography using a 0 to 50% ethyl acetate in petroleum ether gradient to get 40 mg of pure product. 1H NMR (CDCl₃): 7.2 to 7.5 (m, 5H); 6.68 (s, 1H); 5.2 (bs, 2H); 2.95 (s, 3H); 2.2 to 1.5 (m, 14H); 0.9 to 0.6 (m, 24H). ESI-MS: M+H=576.

### Example 17: Synthesis of Carnosic Acid Dipropionate (30)

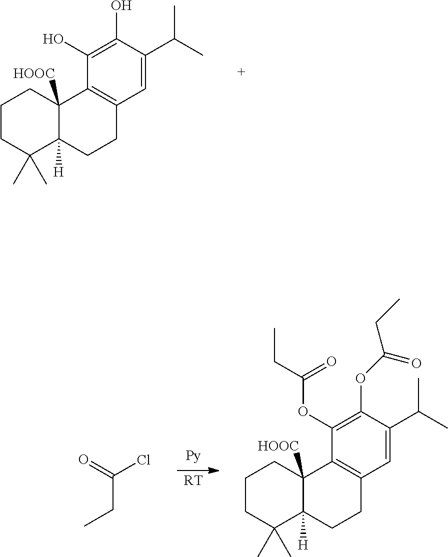

Procedure: To stirred solution of Carnosic Acid (5000 mg, 15 mmol) in anhydrous Pyridine (25 mL), was added 5.57 g of propionyl chloride. The reaction mixture was stirred for 12 h at room temperature. After complete consumption of starting material, the reaction mixture was evaporated to remove 90% of pyridine, re-dissolved in ethyl acetate and washed with water (25 mL×2), saturated bicarbonate solution (25 mL×2) and brine (25 mL×1) dried over sodium sulfate, concentrated to a residue and purified by silica gel column chromatography using a 20 to 100% ethyl acetate in petroleum ether gradient to get 2500 mg of pure product. 1H NMR (CDCl): 6.68 (s, 1H); 2.3 to 1.5 (m, 16H); 0.96 to 0.7 (m, 18H). ESI-MS (negative mode): M−H=443.2.

### Example 18: Synthesis of Compound 24

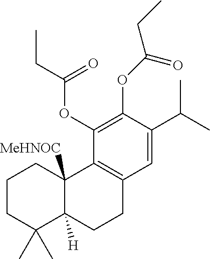

Procedure: To stirred solution of Carnosic Acid Dipropionate (30) (200 mg, 0.449 mmol) in DCM (2 mL), was added oxalyl chloride (114 mg, 0.899 mmol) and one drop of DMF. Reaction mixture (REACTION MIXTURE) was stirred at RT for 1 hr. REACTION MIXTURE was concentrated to residue which is re-dissolved in DCM (5 mL) and added drop wise to a stirred solution of Methyl amine (3 mL, 6.0 mmol, 2M solution in THF) in DCM and TEA (0.324 mL, 2.25 mmol) at 0° C. and stirred for 1 hrs. REACTION MIXTURE was washed with water, 1N HCl solution, bicarbonate and brine solution, organic layer was dried concentrated and purified by silica gel column chromatography using a 0 to 50% ethyl acetate in petroleum ether gradient to get 120 mg of pure product. 1H NMR (CDCl₃): 6.68 (s, 1H); 5.1 (bs, 1H); 3.1 (bs, 3H); 2.3 to 1.5 (m, 16H); 0.96 to 0.7 (m, 18H). ESI-MS: M+H=458.

### Example 19: Synthesis of Compound 25

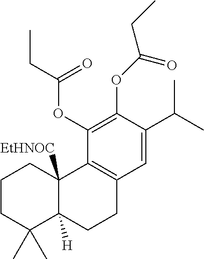

Procedure: To stirred solution of Carnosic Acid Dipropionate (30) (200 mg, 0.449 mmol) in DCM (2 mL), was added oxalyl chloride (114 mg, 0.899 mmol) and one drop of DMF. Reaction mixture (REACTION MIXTURE) was stirred at RT for 1 hr. REACTION MIXTURE was concentrated to residue which is re-dissolved in DCM (5 mL) and added drop wise to a stirred solution of Ethyl amine (2 mL, 2.47 mmol, 60% aqueous solution) in 5 mL DCM and sodium bicarbonate (207 mg, 2.47 mmol) at 0° C. and stirred for 1 hrs. REACTION MIXTURE was washed with water, 1N HCl solution, bicarbonate and brine solution, organic layer was dried concentrated and purified by silica gel column chromatography using a 0 to 50% ethyl acetate in petroleum ether gradient to get 165 mg of pure product. 1H NMR (CDCl₃): 6.68 (s, 1H); 5.1 (bs, 1H); 2.8 (bs, 2H); 2.3 to 1.5 (m, 19H); 0.96 to 0.7 (m, 18H). ESI-MS: M+H=472.

### Example 20: Synthesis of Compound 26

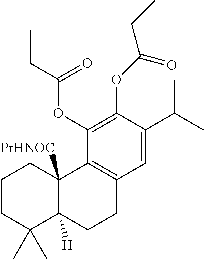

Procedure: To stirred solution of Carnosic Acid Dipropionate (30) (200 mg, 0.449 mmol) in DCM (2 mL), was added oxalyl chloride (114 mg, 0.899 mmol) and one drop of DMF. Reaction mixture (REACTION MIXTURE) was stirred at RT for 1 hr. REACTION MIXTURE was concentrated to residue which is re-dissolved in DCM (5 mL) and added drop wise to a stirred solution of propyl amine (133 mg, 2.25 mmol) in DCM and TEA (0.324 mL, 2.25 mmol) at 0° C. and stirred for 1 hrs. REACTION MIXTURE was washed with water, 1N HCl solution, bicarbonate and brine solution, organic layer was dried concentrated and purified by silica gel column chromatography using a 0 to 50% ethyl acetate in petroleum ether gradient to get 120 mg of pure product. 1H NMR (CDCl₃): 6.68 (s, 1H); 5.1 (bs, 1H); 2.8 (bs, 2H); 2.3 to 1.5 (m, 21H); 0.96 to 0.7 (m, 18H). ESI-MS: M+H=486.

### Example 21: Synthesis of Compound 32

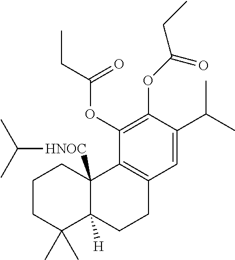

Procedure: To stirred solution of Carnosic Acid Dipropionate (30) (200 mg, 0.449 mmol) in DCM (2 mL), was added oxalyl chloride (114 mg, 0.899 mmol) and one drop of DMF. Reaction mixture (REACTION MIXTURE) was stirred at RT for 1 hr. REACTION MIXTURE was concentrated to residue which is re-dissolved in DCM (5 mL) and added drop wise to a stirred solution of isopropyl amine (142 mg, 2.4 mmol) in DCM and TEA (0.344 mL, 2.40 mmol) at 0° C. and stirred for 1 hrs. REACTION MIXTURE was washed with water, 1N HCl solution, bicarbonate and brine solution, organic layer was dried concentrated and purified by silica gel column chromatography using a 0 to 50% ethyl acetate in petroleum ether gradient to get 152 mg of pure product. 1H NMR (CDCl₃): 6.68 (s, 1H); 5.1 (bs, 1H); 2.8 (m, 1H); 2.3 to 1.5 (m, 19H); 0.96 to 0.7 (m, 21H). ESI-MS: M+H=487.

### Example 22: Synthesis of Compound 27

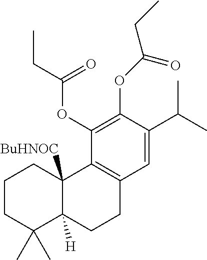

Procedure: To stirred solution of Carnosic Acid Dipropionate (30) (200 mg, 0.449 mmol) in DCM (2 mL), was added oxalyl chloride (114 mg, 0.899 mmol) and one drop of DMF. Reaction mixture (REACTION MIXTURE) was stirred at RT for 1 hr. REACTION MIXTURE was concentrated to residue which is re-dissolved in DCM (5 mL) and added drop wise to a stirred solution of butyl amine (164 mg, 2.25 mmol) in DCM and TEA (0.324 mL, 2.25 mmol) at 0° C. and stirred for 1 hrs. REACTION MIXTURE was washed with water, 1N HCl solution, bicarbonate and brine solution, organic layer was dried concentrated and purified by silica gel column chromatography using a 0 to 50% ethyl acetate in petroleum ether gradient to get 150 mg of pure product. 1H NMR (CDCl₃): 6.68 (s, 1H); 5.1 (bs, 1H); 2.8 (bs, 2H); 2.3 to 1.5 (m, 23H); 0.96 to 0.7 (m, 18H). ESI-MS: M+H=500.

### Example 23: Synthesis of Compound 28

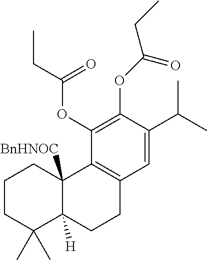

Procedure: To stirred solution of Carnosic Acid Dipropionate (30) (200 mg, 0.449 mmol) in DCM (2 mL), was added oxalyl chloride (114 mg, 0.899 mmol) and one drop of DMF. Reaction mixture (REACTION MIXTURE) was stirred at RT for 1 hr. REACTION MIXTURE was concentrated to residue which is re-dissolved in DCM (5 mL) and added drop wise to a stirred solution of benzyl amine (285 mg, 2.25 mmol) in DCM and TEA (0.324 mL, 2.25 mmol) at 0° C. and stirred for 1 hrs. REACTION MIXTURE was washed with water, 1N HCl solution, bicarbonate and brine solution, organic layer was dried concentrated and purified by silica gel column chromatography using a 0 to 50% ethyl acetate in petroleum ether gradient to get 160 mg of pure product. 1H NMR (CDCl₃): 7.2 to 7.6 (m, 5H); 6.68 (s, 1H); 5.2 (bs, 2H); 5.1 (bs, 1H); 2.3 to 1.5 (m, 16H); 0.96 to 0.7 (m, 18H). ESI-MS: M+H=534.

### Example 24: Synthesis of Compound 29

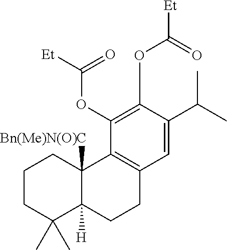

Procedure: To stirred solution of Carnosic Acid Dipropionate (30) (200 mg, 0.449 mmol) in DCM (2 mL), was added oxalyl chloride (114 mg, 0.899 mmol) and one drop of DMF. Reaction mixture (REACTION MIXTURE) was stirred at RT for 1 hr. REACTION MIXTURE was concentrated to residue which is re-dissolved in DCM (5 mL) and added drop wise to a stirred solution of N-Methyl Benzyl amine (207 mg, 2.25 mmol) in DCM and TEA (0.324 mL, 2.25 mmol) at 0° C. and stirred for 1 hrs. REACTION MIXTURE was washed with water, 1N HCl solution, bicarbonate and brine solution, organic layer was dried concentrated and purified by silica gel column chromatography using a 0 to 50% ethyl acetate in petroleum ether gradient to get 45 mg of pure product. 1H NMR (CDCl₃): 7.2 to 7.6 (m, 5H); 6.68 (s, 1H); 5.2 (bs, 2H); 3.15 (s, 3H); 2.3 to 1.5 (m, 16H); 0.96 to 0.7 (m, 18H). ESI-MS: M+H=548.

### Example 25: Synthesis of Compound 18

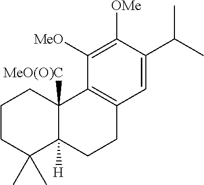

Procedure: Carnosic acid (250 mg, 0.752 mmol) was dissolved in 2 mL of DMF, evacuated and back purged with argon, followed by addition of potassium carbonate (519 mg, 3.76 mmol) and methyl iodide (533 mg, 3.76 mmol). The reaction mixture was stirred at room temperature for 12 h at which time TLC analysis indicated completion of reaction. The reaction mixture was diluted with 15 mL of ethyl acetate and washed with water (5 mL×3), saturated sodium bicarbonate solution (5 mL×2) and brine. The combined organic phase was dried over sodium sulphate and concentrated. The residue was purified by silica gel column chromatography using a gradient 0 to 35% ethyl acetate in petroleum ether to yield 50 mg of desired product. 1H NMR (CDCl₃): 6.68 (s, 1H); 3.75 (s, 3H); 3.66 (s, 3H); 3.65 (s, 3H); 3.4 (m, 1H); 3.24 (m, 1H); 2.84 (m, 2H); 2.22 (m, 2H); 1.8 (m, 1H); 1.52 (t, J=12 Hz, 3H); 1.3 to 1.16 (m, 8H); 0.97 (s, 3H); 0.77 (s, 3H). ESI, M+H=375.

### Example 26: Synthesis of Compound 19

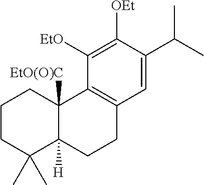

Procedure: Carnosic acid (250 mg, 0.752 mmol) was dissolved in 2 mL of DMF, evacuated and back purged with argon, followed by addition of potassium carbonate (519 mg, 3.76 mmol) and ethyl iodide (586 mg, 3.76 mmol). The reaction mixture was stirred at room temperature for 12 h at which time TLC analysis indicated completion of reaction. The reaction mixture was diluted with 15 mL of ethyl acetate and washed with water (5 mL×3), saturated sodium bicarbonate solution (5 mL×2) and brine. The combined organic phase was dried over sodium sulphate and concentrated. The residue was purified by silica gel column chromatography using a gradient 0 to 35% ethyl acetate in petroleum ether to yield 90 mg of desired product. 1H NMR (CDCl₃): 6.68 (s, 1H); 3.75 to 3.65 (m, 6H); 3.4 (m, 1H); 3.24 (m, 1H); 2.84 (m, 2H); 2.22 (m, 2H); 1.8 (m, 1H); 1.52 (t, J=12 Hz, 3H); 1.3 to 0.77 (m, 23H). ESI, M+H=417.

### Example 27: Synthesis of Compound 20

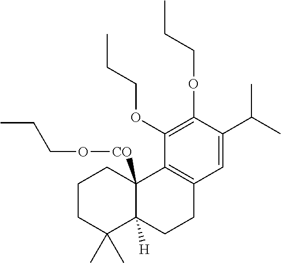 [SMILES: CCCOC(=O)[C@]12CCCC(C)(C)[C@@H]1CCc1cc(C(C)C)c(OCCC)c(OCCC)c12]

Procedure: Carnosic acid (250 mg, 0.752 mmol) was dissolved in 2 mL of DMF, evacuated and back purged with argon, followed by addition of potassium carbonate (519 mg, 3.76 mmol) and propyl iodide (639 mg, 3.76 mmol). The reaction mixture was stirred at room temperature for 12 h at which time TLC analysis indicated completion of reaction. The reaction mixture was diluted with 15 mL of ethyl acetate and washed with water (5 mL×3), saturated sodium bicarbonate solution (5 mL×2) and brine. The combined organic phase was dried over sodium sulphate and concentrated. The residue was purified by silica gel column chromatography using a gradient 0 to 35% ethyl acetate in petroleum ether to yield 105 mg of desired product. 1H NMR (CDCl₃): 6.68 (s, 1H); 3.75 to 3.65 (m, 6H); 3.4 (m, 1H); 3.24 (m, 1H); 2.84 (m, 2H); 2.22 (m, 2H); 1.8 (m, 1H); 1.52 (t, J=12 Hz, 3H); 1.3 to 0.77 (m, 27H). ESI, M+H=459.

### Example 28: Synthesis of Compound 23

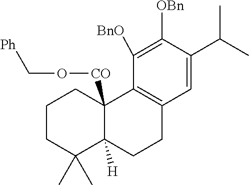

Procedure: Carnosic acid (250 mg, 0.752 mmol) was dissolved in 2 mL of DMF, evacuated and back purged with argon, followed by addition of potassium carbonate (519 mg, 3.76 mmol) and benzyl bromide (643 mg, 3.76 mmol). The reaction mixture was stirred at room temperature for 12 h at which time TLC analysis indicated completion of reaction. The reaction mixture was diluted with 15 mL of ethyl acetate and washed with water (5 mL×3), saturated sodium bicarbonate solution (5 mL×2) and brine. The combined organic phase was dried over sodium sulphate and concentrated. The residue was purified by silica gel column chromatography using a gradient 0 to 35% ethyl acetate in petroleum ether to yield 150 mg of desired product. 1H NMR (CDCl₃): 7.2 to 7.7 (m, 15H); 6.68 (s, 1H); 5.2 to 5.4 (m, 6H); 3.4 (m, 1H); 3.24 (m, 1H); 2.84 (m, 2H); 2.22 (m, 2H); 1.8 (m, 1H); 1.52 (t, J=12 Hz, 3H); 1.3 to 1.16 (m, 8H); 0.97 (s, 3H); 0.77 (s, 3H). ESI, M+H=603.

### Example 29: Synthesis of Compound 22

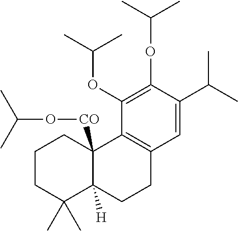 [SMILES: CC(C)OC(=O)[C@]12CCCC(C)(C)[C@@H]1CCc1cc(C(C)C)c(OC(C)C)c(OC(C)C)c12]

Procedure: Carnosic acid (250 mg, 0.752 mmol) was dissolved in 2 mL of DMF, evacuated and back purged with argon, followed by addition of potassium carbonate (519 mg, 3.76 mmol) and isopropyl iodide (639 mg, 3.76 mmol). The reaction mixture was stirred at room temperature for 12 h at which time TLC analysis indicated completion of reaction. The reaction mixture was diluted with 15 mL of ethyl acetate and washed with water (5 mL×3), saturated sodium bicarbonate solution (5 mL×2) and brine. The combined organic phase was dried over sodium sulphate and concentrated. The residue was purified by silica gel column chromatography using a gradient 0 to 35% ethyl acetate in petroleum ether to yield 105 mg of desired product. 1H NMR (CDCl₃): 6.68 (s, 1H); 3.75 to 3.5 (m, 3H); 3.4 (m, 1H); 3.24 (m, 1H); 2.84 (m, 2H); 2.22 (m, 2H); 1.8 (m, 1H); 1.52 (t, J=12 Hz, 3H); 1.3 to 1.16 (m, 8H); 1.0 to 0.60 (s, 24H). ESI, M+H=459.

### Example 30: Synthesis of Compound 21

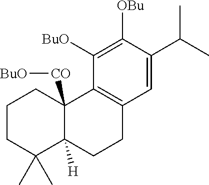

Procedure: Carnosic acid (250 mg, 0.752 mmol) was dissolved in 2 mL of DMF, evacuated and back purged with argon, followed by addition of potassium carbonate (519 mg, 3.76 mmol) and n-butyl bromide (515 mg, 3.76 mmol). The reaction mixture was stirred at room temperature for 12 h at which time TLC analysis indicated completion of reaction. The reaction mixture was diluted with 15 mL of ethyl acetate and washed with water (5 mL×3), saturated sodium bicarbonate solution (5 mL×2) and brine. The combined organic phase was dried over sodium sulphate and concentrated. The residue was purified by silica gel column chromatography using a gradient 0 to 35% ethyl acetate in petroleum ether to yield 100 mg of desired product. 1H NMR (CDCl₃): 6.68 (s, 1H); 3.75 to 3.65 (m, 6H); 3.4 (m, 1H); 3.24 (m, 1H); 2.84 (m, 2H); 2.22 (m, 2H); 1.8 to 1.4 (m, 16H); 1.3 to 0.77 (m, 27H). ESI, M+H=501.

### Example 31: Synthesis of Compound 17

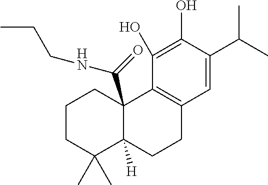 [SMILES: CCCNC(=O)[C@]12CCCC(C)(C)[C@@H]1CCc1cc(C(C)C)c(O)c(O)c12]

Procedure: To stirred solution of Carnosic propyl amide diacetate (3) (100 mg, 0.218 mmol), THF (2 mL) at 0° C., was added LAH (42 mg) portion wise. REACTION MIXTURE was stirred for 1 hrs. TLC checked. REACTION MIXTURE was quenched by addition of sat. ammonium chloride solution and extracted with Ethyl acetate. Dried, concentrated and purified by silica gel column chromatography to yield 55 mg of carnosic propyl amide. 1H NMR (CDCl₃): 6.97 (s, 1H); 5.10 (bs, 1H); 3.20 (m, 2H); 2.92 (m, 3H); 2.73 (m, 1H); 2.60 (m, 2H); 2.2-1.0 (m, 17H); 0.96 (s, 3H); 0.82 (s, 3H). ESI, M+H=374.

### Example 32: Synthesis of Compound 16

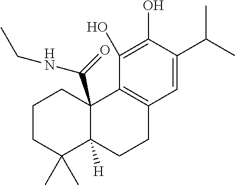 [SMILES: CCNC(=O)[C@]12CCCC(C)(C)[C@@H]1CCc1cc(C(C)C)c(O)c(O)c12]

Procedure: To stirred solution of Carnosic ethyl amide diacetate (2) (200 mg, 0.450 mmol), THF (4 mL) at 0° C., was added LAH (86 mg) portion wise. REACTION MIXTURE was stirred for 1 hrs. TLC checked. REACTION MIXTURE was quenched by addition of sat. ammonium chloride solution and extracted with Ethyl acetate. Dried, concentrated and purified by silica gel column chromatography to yield 155 mg of carnosic ethyl amide. 1H NMR (CDCl₃): 6.97 (s, 1H); 5.10 (bs, 1H); 3.20 (m, 2H); 2.92 (m, 3H); 2.73 (m, 1H); 2.60 (m, 2H); 2.2-1.0 (m, 15H); 0.96 (s, 3H); 0.82 (s, 3H). ESI, M+H=360.

### Example 33: Synthesis of Compound 15

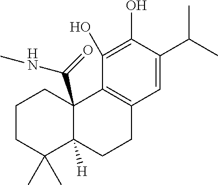 [SMILES: CNC(=O)[C@]12CCCC(C)(C)[C@@H]1CCc1cc(C(C)C)c(O)c(O)c12]

Procedure: To stirred solution of Carnosic Methyl amide diacetate (1) (200 mg, 0.465 mmol), THF (4 mL) at 0° C., was added LAH (88 mg) portion wise. REACTION MIXTURE was stirred for 1 hrs. TLC checked. REACTION MIXTURE was quenched by addition of sat. ammonium chloride solution and extracted with Ethyl acetate. Dried, concentrated and purified by silica gel column chromatography to yield 100 mg of carnosic methyl amide. 1H NMR (CDCl₃): 6.97 (s, 1H); 5.10 (bs, 1H); 3.20 (m, 2H); 2.92 (m, 3H); 2.73 (m, 1H); 2.60 (d, 3H); 2.2-1.0 (m, 12H); 0.96 (s, 3H); 0.82 (s, 3H). ESI, M+H=346.

### Part II: Biological Examples

Proinflammatory microglial activation. Following a protocol adapted from D. Trudler et al. (2021), human induced pluripotent stem cell (hiPSC)-derived microglia (hiMG) were plated at a concentration of 1×10⁶ cells/mL. 16 h before the experiment the cell medium was changed to IMDM (no supplements added). Carnosic acid (CA, purity >99%) was added 16 hours before the experiment, at a concentration of 2 μM. Recombinant αSyn oligomers (100 nM) and oligomeric Aβ (10 μM) were incubated with 2.5 μg/mL of their respective antibodies for 1 h prior to adding to hiMG for additional 6 hours. Media was collected and cytokine levels, IL-1β or tumor necrosis factor (TNF), were measured by ELISA per the manufacturer's instructions.

Preparation of Humanized scFv Against αSyn. scFv antibody has been previously generated and characterized (Emadi et al., 2004). scFv was expressed and isolated as previously described with some modifications (Emadi et al., 2007). Briefly, soluble scFv was produced by expressing recovered phagemid samples in the non-suppressor E. coli strain HB2151. scFv production was induced by addition of 1 mM isopropyl-β-D-thiogalactopyranoside (IPTG, Sigma) and incubated overnight at 30° C. The supernatant and periplasmic fractions were combined and applied to HisPur Cobalt Resin (Thermo-Fisher Scientific, #90091), eluted with 10-25 mM imidazole and buffer exchanged to PBS (Thermo-Fisher Scientific, #90011). The eluates were quantified with a Pierce™ BCA Protein Assay Kit (Thermo-Fisher Scientific, #23225).

Preparation of Humanized Antibody Against Aβ. The single chain antibody scFv against panAβ was previously generated and characterized (Levites et al., 2006). Here, anti-pan Aβ scFv was fused to human Fc1 to create a minibody scFv-Fc. Minibodies were expressed in HEK293 cells for 48 h and isolated from the conditioned medium using HisPur Cobalt Resin (Thermo-Fisher Scientific, #PI90092), eluted with 10-25 mM imidazole, and buffer exchanged to PBS (Thermo-Fisher Scientific, #PI87773). The eluates were quantified with a Pierce™ BCA Protein Assay Kit (Thermo-Fisher Scientific, #23225), and analyzed for purity on a gel with an In Vision™ His-Tag In-Gel Staining Kit (Thermo-Fisher Scientific, #LC6033).

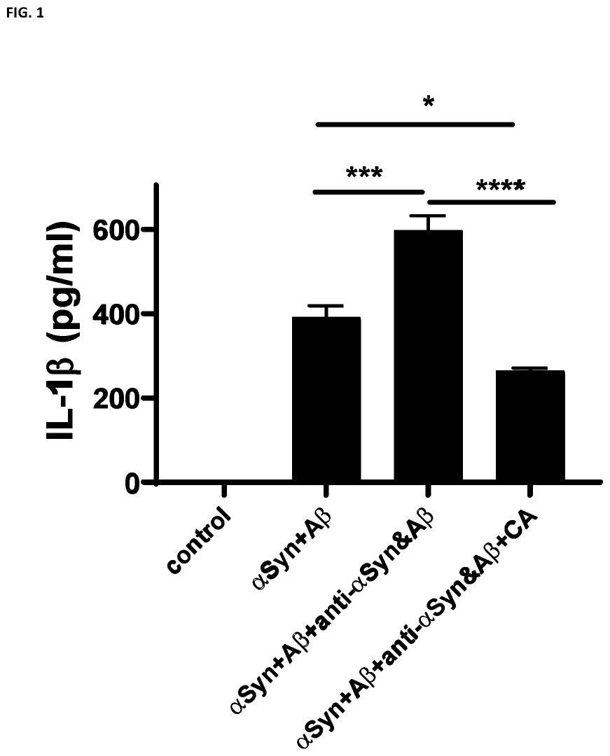

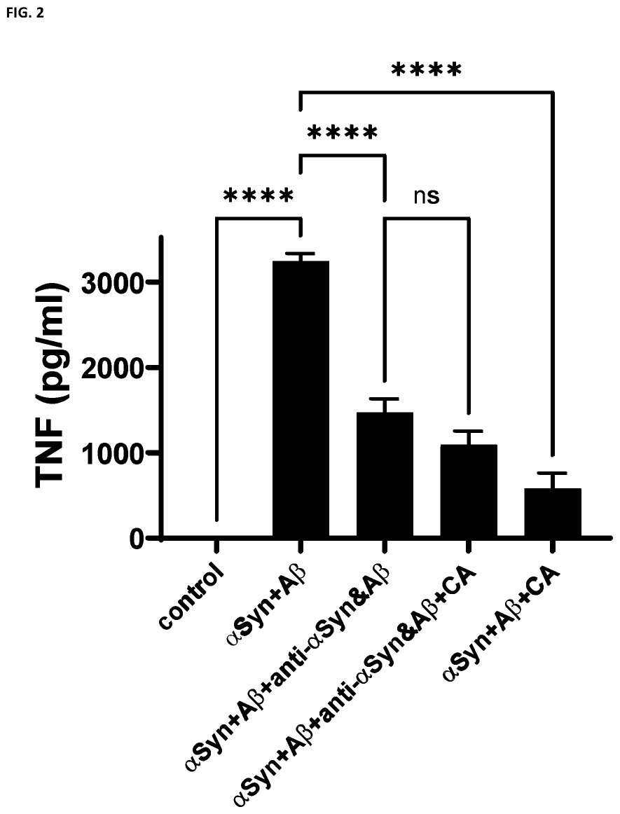

Carnosic Acid Reduces Neuroinflammation Induced by Antibody-Misfolded Protein Complex. NLRP3 inflammasome activation was assessed by measuring the release of the proinflammatory cytokine interleukin-1β (IL-1β) from hiMG exposed to (A) oligomeric Aβ (500 nM oligomers) and low concentrations of oligomeric αSyn (100 nM), (B) Aβ and αSyn with their cognate human antibodies (anti-Aβ and anti-αSyn, respectively), and (C) same as (B) with carnosic acid. Carnosic acid (0.1-10 μM) significantly reduced the type of inflammation caused by formation of anti-Aβ and anti-αSyn complexes, as evidenced by the reduction in IL-1β (FIG. 1) and TNF (FIG. 2) levels.

### REFERENCES CITED IN EXAMPLES

Emadi, S., Barkhordarian, H., Wang, M. S., Schulz, P., and Sierks, M. R. (2007). Isolation of a human single chain antibody fragment against oligomeric α-synuclein that inhibits aggregation and prevents α-synuclein-induced toxicity. J Mol Biol 368, 1132-1144.
Emadi, S., Liu, R., Yuan, B., Schulz, P., McAllister, C., Lyubchenko, Y., Messer, A., and Sierks, M. R. (2004). Inhibiting aggregation of α-synuclein with human single chain antibody fragments. Biochemistry 43, 2871-2878.
Levites, Y., Jansen, K., Smithson, L. A., Dakin, R., Holloway, V. M., Das, P., and Golde, T. E. (2006). Intracranial adeno-associated virus-mediated delivery of anti-pan Aβ, Aβ40, and Aβ42 single-chain variable fragments attenuates plaque pathology in amyloid precursor protein mice. J Neurosci 26, 11923-11928.

We claim:

1. A method for treating a neurodegenerative disease in a subject suffering therefrom, comprising administering to the subject:
(A) at least one antibody that is directed against an aberrant misfolded protein; and
(B) at least one compound or pharmaceutically acceptable salt thereof that activates the nuclear factor (erythroid-derived 2)-like 2 (Nrf2) and/or heat-shock factor-1 (HSF-1) transcription-mediated signaling pathway.

2. The method according to claim 1, wherein the antibody (A) is administered before the compound (B).

3. The method according to claim 1, wherein the antibody (A) is administered simultaneously with the compound (B).

4. The method according to claim 1, wherein the antibody (A) is administered after the compound (B).

5. A method for treating neuroinflammation in a subject suffering therefrom, wherein the subject is one who:
(a) suffers from a neurodegenerative disease, and/or
(b) has been administered at least one antibody that is directed against an aberrant misfolded protein;
the method comprising administering to the subject at least one compound or pharmaceutically acceptable salt thereof that activates the nuclear factor (erythroid-derived 2)-like 2 (Nrf2) and/or heat-shock factor-1 (HSF-1) transcription-mediated signaling pathway.

6. The method according to claim 5, wherein the subject is one who (a) suffers from a neurodegenerative disease.

7. The method according to claim 5, wherein the subject is one who (b) has been administered at least one antibody that is directed against an aberrant misfolded protein.

8. The method according to claim 5, wherein the subject is one who (a) suffers from a neurodegenerative disease and (b) has been administered at least one antibody that is directed against an aberrant misfolded protein.

9. The method according to any one of claims 1 to 8, wherein the aberrant misfolded protein is chosen from amyloid-beta (Aβ), alpha-synuclein (αSYN), another misfolded protein to which there is a cognate antibody therapy, and a combination thereof.

10. The method according to any one of claims 1 to 8, wherein the aberrant misfolded protein is Aβ.

11. The method according to any one of claims 1 to 8, wherein the aberrant misfolded protein is αSYN.

12. The method according to any one of claims 1 to 8, wherein the aberrant misfolded protein is a combination of Aβ and αSYN.

13. The method according to any one of claims 1 to 12, wherein the neurodegenerative disease is chosen from Alzheimer's disease, Parkinson's disease, and Lewy body dementia, and another degenerative disease with a misfolded protein that contributes to the condition.

14. The method according to any one of claims 1 to 13, wherein the compound is one selected from the following table:
Carnosic acid
Para carnosic acid
Yahazunol
(+)-yahazunol
Zonarone
Zonarol
Isozonarone
Isozonarol
Cyclozonarone
Spongiaquinone
Hyatellaquinone
20-O-Acetyl-21-hydroxy-(−)-isozonarol
Avarol
Avarone
20-O-Acetylneoavarol
Ilimaquinone
Isospongiaquinone
5-Epi-ilimaquinone
5-Epi-isospongiaquinone
Arenarol
Puupehenone
4′-Isobutylthioavarone
3′-Methoxyavarone
Metachromins A
Metachromins B
Hypochromin A diacetate
Metachromin B monoacetate
Neomarinone
Strongylophorine
Puupehedione
Puupehediol
3′,4′-Ethylenedithioavarone
Isoarenarol
Melemeleone B
2-Epi-Nakijiquinone
FrondosinA
Peyssonols A
Peyssonols B
2-Hexaprenylhydroquinone
Dimethoxypuupehenol
Isoavarone
Nakijiquinone A
Nakijiquinone B
Nakijiquinone C
Nakijiquinone D
Nakijinol
Nakijiquinone E
Nakijiquinone F
Nakijiquinone G
Nakijiquinone H
Nakijiquinone I
Nakijiquinone J
Nakijiquinone K
Nakijiquinone L
Nakijiquinone M
Nakijiquinone N
Nakijiquinone O
Nakijiquinone P
Nakijiquinone Q
Nakijiquinone R
Polyfibrospongol A
Polyfibrospongol B
19-Hydroxy-polyfibrospongol B
Smenospondiol
5-Epi-Ilimaquinone
Ilimaquinone
Glycinylilimaquinone
Smenosquinone
Smenospongidine
(+)-Epi-Smenospongidin
Smenospongiarine
(+)-Epi-Smenospongiarin
Smenospongorine
5-Epi-Smenospongorine
Smenorthoquinone
Smenospongin
Cyclosmenospongine
Avarol
Neoavarol
6′-Hydroxyavarol
6′-Acetoxyavarol
Monoacetylavarol
6′-Hydroxy-5′-acetylavarol
Diacetylavarol
2′,5′-0-(4-Bromobenzoyl)avarol
Arenarol
Avarone
Neoavarone
4′-Methoxyavarone
4′-Methoxyneoavarone
3′-Hydroxyavarone
6′-Acetoxyavarone
3′,6′-Dihydroxyavarone
6′-Hydroxy-4′-methoxyavarone
4′-Methylaminoavarone
3′,4′-(Ethylenedithio)avarone
Melemeleone A
Melemeleone B
Arenarone
Avinosol
Wiedendiol A
Wiedendiol B
Corallidyctal A
Corallidyctal B
Corallidyctal C
Corallidyctal D
Chromazonarol
(+)-Chromazonarol
8-Epi-Chromazonarol
Puupehenol
15α-methoxypuupehenol
Cyanopuupehenone
Puupehenone
Puupehenone
Puupehenione
Cyclosiphonodictyol bis-sulfate A
Mamanuthaquinone
Smenoqualone
Strongyline A
Bolinaquinone
Dehydroxybolinaquinone
Dysidine
Methoxyhalenaquinone
Fulvanin-2
Panicein A2
Panicein F1
Renierin A
Renierin B
Popolohuanone A
Popolohuanone B
Popolohuanone C
Popolohuanone D
Dipuupehenone
Bispuupehenone
Popolohuanone E
popolohuanone F
or a pharmaceutically acceptable salt thereof.

15. The method according to any one of claims 1 to 14, wherein the compound is carnosic acid or a pharmaceutically acceptable salt thereof.

16. The method according to any one of claims 1 to 13, wherein the compound is of Formula (I): 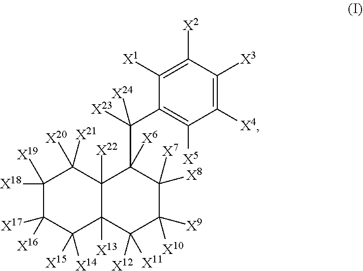
wherein
each of X¹ to X²⁴ is independently selected from the group consisting of H, OR¹ (wherein R¹ is H or C₁-C₆-alkyl), C₁-C₆-alkyl, and W, wherein
each W independently is of the formula —B—C-D, —C—B-D, or —C—B—C-D;
B is selected from the group consisting of a bond, —C(O)—, —C(O)O—, —O—, —SO₂—, —NH—, —NHC(O)—, and —C(O)—NH—;
C is selected from the group consisting of a bond, and mono- or divalent C₁-C₆-alkyl, C₃-C₁₀-cycloalkyl, C₂-C₆-alkenyl, C₃-C₁₀-cycloalkenyl, C₆-C₁₀-aryl, —C₁-C₆-alkyl-(C₆-C₁₀-aryl), and —C₂-C₆-alkenyl-(C₆-C₁₀-aryl);
wherein C is optionally substituted with one to three substituents selected from the group consisting of C₁-C₆-alkyl, C₁-C₆-haloalkyl, C₁-C₆-alkoxy, C₁-C₆-haloalkoxy, CN, OH, NO₂, —NRR′ (wherein R and R′ are independently selected from H and C₁-C₆-alkyl), —(C₁-C₆-alkyl)NRR′, —C(O)NRR′, —SO₂R, C₆-C₁₀-aryl, 3- to 6-membered heterocycloalkyl (wherein 1-4 ring members are independently selected from N, O, and S), 5- to 10-membered heteroaryl (wherein 1-4 heteroaryl members are independently selected from N, O, and S), and C₃-C₁₀-cycloalkyl
D is selected from the group consisting of H, —C(O)OR¹, —SO₃H, —OP(O)(OR)(OR′), —NO₃, —NO₂, —NO, —NH₂, and —OH;
optionally one pair of X⁷/X⁸, X⁹/X¹⁰, X¹¹/X¹², X¹⁴/X¹⁵, X¹⁶/X¹⁷, X¹⁸/X¹⁹, X²⁰/X²¹, and X²³/X²⁴ represents ═C(R¹)₂,
OR, optionally, one pair of substituents X⁶/X⁷, X⁸/X⁹, X¹⁰/X¹¹, X¹²/X¹³, X¹³/X¹⁴, X¹⁵/X¹⁶, X¹⁷/X¹⁸, X¹⁹X²⁰, X²¹/X²², X⁶/X²², X¹³/X²², and X⁶/X²³, together with the bond between adjacent carbon atoms to which the substituents are bound, represents a double bond;
or a pharmaceutically acceptable salt thereof.

17. The method according to claim 16, wherein X¹ and X⁴ are independently OR¹.

18. The method according to claim 17, wherein each R¹ is H.

19. The method according to any one of claims 16 to 18, wherein one pair of X¹/X⁸, X⁹/X¹⁰, X¹¹/X¹², X¹⁴/X¹⁵, X¹⁶/X¹⁷, X¹⁸/X¹⁹, X²⁰/X²¹, and X²³/X²⁴ represents ═C(R¹)₂.

20. The method according to any one of claims 16 to 18, wherein one pair of substituents X⁶/X⁷, X⁸/X⁹, X¹⁰/X¹¹, X¹²/X¹³, X¹³/X¹⁴, X¹⁵/X¹⁶, X¹⁷/X¹⁸, X¹⁹X²⁰, X²¹/X²², X⁶/X²², X¹³/X²², and X⁶/X²³, together with the bond between adjacent carbon atoms to which the substituents are bound, represents a double bond.

21. A compound of Formula (II): 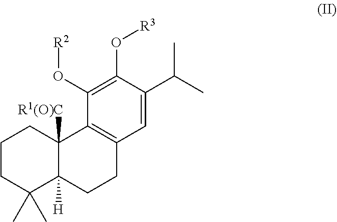
wherein
R¹ is selected from the group consisting of —NR^{A}R^{B}, —OR^{A}, —OP(O)(OR^{A})(OR^{B});
R² and R³ are independently selected from the group consisting of H, —C(O)R^{A}, —OR^{A}, —C(O)NR^{A}R^{B}, and —OP(O)(OR^{A})(OR^{B});
R^{A} and R^{B} are independently selected from H, C₁-C₆-alkyl, C₁-C₆-alkyl-(C₆-C₁₀-aryl);
and wherein when R¹ is —OH, then R² and R³ are not simultaneously H;
or a pharmaceutically acceptable salt thereof.

22. The compound according to claim 21, wherein R² and R³ are independently-C(O)R^{A}.

23. The compound according to claim 21, wherein R² and R³ are independently R^{A}.

24. The compound according to claim 21, wherein R² and R³ are independently —C(O)NR^{A}R^{B}.

25. The compound according to any one of claims 21 to 24, wherein R¹ is NR^{A}R^{B}.

26. The compound according to any one of claims 21 to 24, wherein R¹ is OR^{A}.

27. The compound according to claim 21, wherein the compound or pharmaceutically acceptable salt thereof is one selected from the following table:

| | |
|---|---|
| 1 | 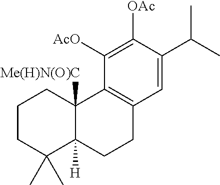 |
| 2 | 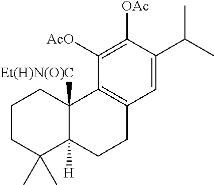 |
| 3 | 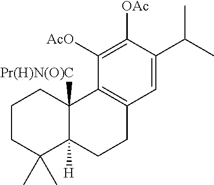 |
| 4 | 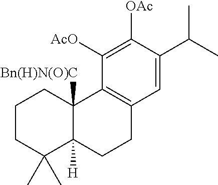 |
| 5 | 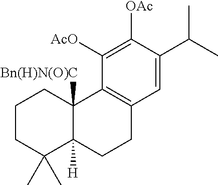 |
| 6 | 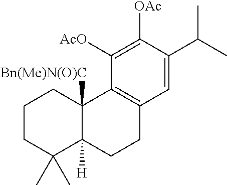 |
| 7 | 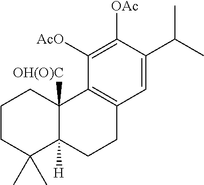 |
| 8 | 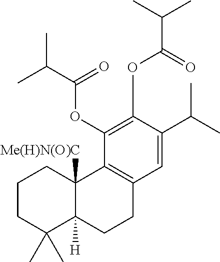 |
| 9 | 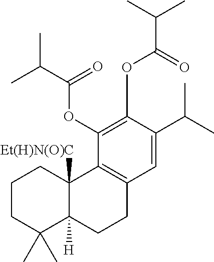 |
| 10 | 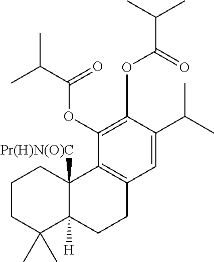 |
| 11 | 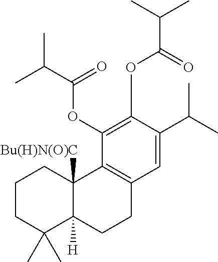 |
| 12 | 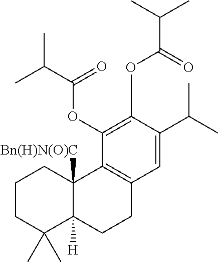 |
| 13 | 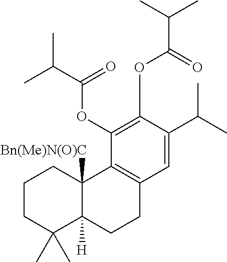 |
| 14 | 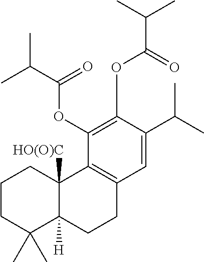 |
| 15 | 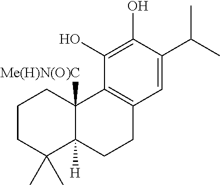 |
| 16 | 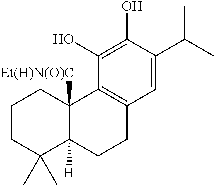 |
| 17 | 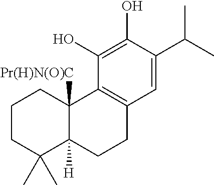 |
| 18 | 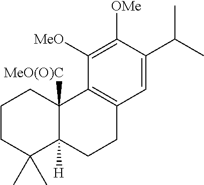 |
| 19 | 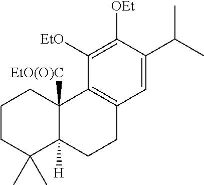 |
| 20 | 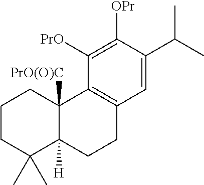 |
| 21 | 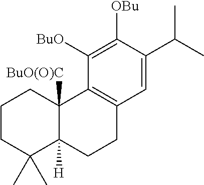 |
| 22 | 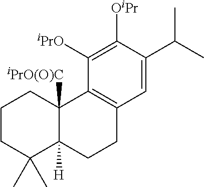 |
| 23 | 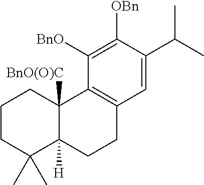 |
| 24 | 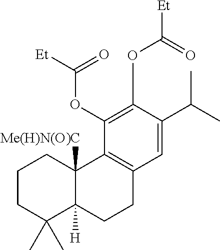 |
| 25 | 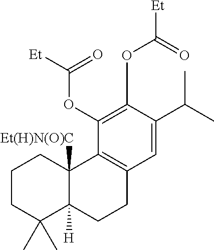 |
| 26 | 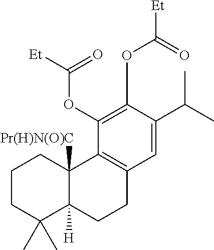 |
| 27 | 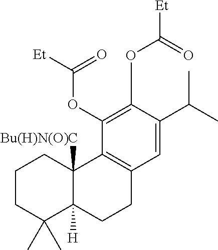 |
| 28 | 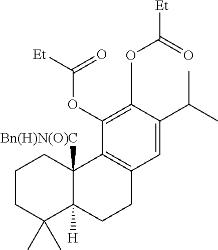 |
| 29 | 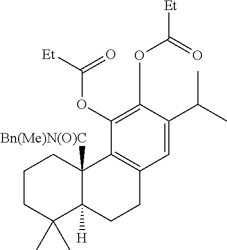 |
| 30 | 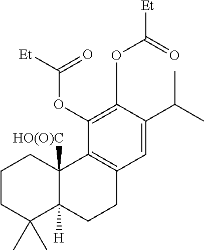 |
| 31 | 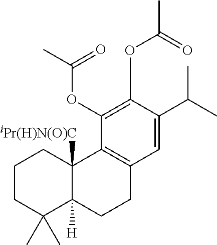 |
| 32 | 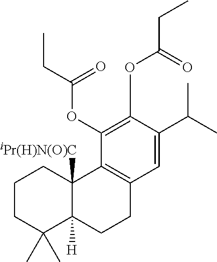 |
| 33 | 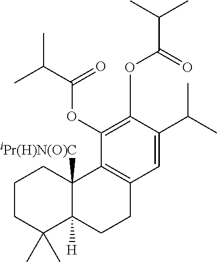 |
| 34 | 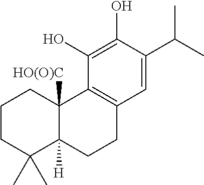 |

28. A pharmaceutical composition comprising a compound or pharmaceutically acceptable salt thereof according to any one of claims 21 to 27 and a pharmaceutically acceptable carrier.

29. A method for treating a neurodegenerative disease in a subject suffering therefrom, comprising administering to the subject:
(A) at least one antibody that is directed against an aberrant misfolded protein; and
(B) at least one compound or pharmaceutically acceptable salt thereof according to any one of claims 21 to 27.

30. The method according to claim 29, wherein the antibody (A) is administered before the compound (B).

31. The method according to claim 29, wherein the antibody (A) is administered simultaneously with the compound (B).

32. The method according to claim 29, wherein the antibody (A) is administered after the compound (B).

33. A method for treating neuroinflammation in a subject suffering therefrom, wherein the subject is one who:
(a) suffers from a neurodegenerative disease, and/or
(b) has been administered at least one antibody that is directed against an aberrant misfolded protein;
the method comprising administering to the subject at least one compound or pharmaceutically acceptable salt thereof according to any one of claims 21 to 27.

34. The method according to claim 33, wherein the subject is one who (a) suffers from a neurodegenerative disease.

35. The method according to claim 33, wherein the subject is one who (b) has been administered at least one antibody that is directed against an aberrant misfolded protein.

36. The method according to claim 33, wherein the subject is one who (a) suffers from a neurodegenerative disease and (b) has been administered at least one antibody that is directed against an aberrant misfolded protein.

37. The method according to any one of claims 29 to 36, wherein the aberrant misfolded protein is chosen from amyloid-beta (Aβ), alpha-synuclein (αSYN), another misfolded protein to which there is a cognate antibody therapy, and a combination thereof.

38. The method according to any one of claims 29 to 36, wherein the aberrant misfolded protein is Aβ.

39. The method according to any one of claims 29 to 36, wherein the aberrant misfolded protein is αSYN.

40. The method according to any one of claims 29 to 36, wherein the aberrant misfolded protein is a combination of Aβ and αSYN.

41. The method according to any one of claims 29 to 40, wherein the neurodegenerative disease is chosen from Alzheimer's disease, Parkinson's disease, and Lewy body dementia, and another degenerative disease with a misfolded protein that contributes to the condition.
